(19) United States
(12) Patent Application Publication
(10) Pub. No.: US 2025/0129045 A1
(43) Pub. Date: Apr. 24, 2025

(54) SMALL MOLECULE PIM AND MTOR KINASE INHIBITOR AND METHODS OF USE THEREOF

(71) Applicant: Arizona Board of Regents on Behalf of the University of Arizona, Tucson, AZ (US)

(72) Inventors: Andrew Kraft, Tucson, AZ (US); Koichi Okumura, Tucson, AZ (US); Wei Wang, Tucson, AZ (US); Giri Gnawali, Tucson, AZ (US)

(21) Appl. No.: 18/835,778

(22) PCT Filed: Feb. 1, 2023
(86) PCT No.: PCT/US2023/061726

§ 371 Date: Aug. 5, 2024

Related U.S. Application Data
(60) Provisional application No. 63/267,563, filed on Feb. 4, 2022.

(51) Int. Cl. C07D 401/12; A61K 31/4545; A61K 31/4704; A61K 31/4709; A61K 31/519; A61K 31/5377; A61K 45/06; C07D 215/227

(52) U.S. Cl. CPC C07D 401/12; A61K 31/4545; A61K 31/4704; A61K 31/4709; A61K 31/519; A61K 31/5377; A61K 45/06; C07D 215/227

(57) ABSTRACT

In one aspect, the disclosure relates to small molecules useful as inhibitors of Pim and mTOR protein kinases, pharmaceutical compositions comprising the same, and methods of treating cancers associated with phosphorylation of protein Enhancer of Decapping 3 (EDC3) including, but not limited to, prostate cancer, as well as other cancers in which Pim and mTOR protein kinases are implicated, using the same. Also disclosed are combination therapies including at least one disclosed small molecule inhibitor and at least one AKT inhibitor.

### CROSS-REFERENCE TO RELATED APPLICATIONS

This application claims the benefit of U.S. Provisional Application No. 63/267,563, filed on Feb. 4, 2022, which is incorporated herein by reference in its entirety.

### BACKGROUND

Prostate cancer (PCa) is the second most common cause of cancer in American men, with nearly 250,000 cases diagnosed annually and with about 34,000 annual prostate cancer deaths. A variety of treatment options exist depending on cancer stage, metastasis, and related factors. However, many treatments have drawbacks. For example, radiation therapy and cryotherapy can both interfere with bowel function and/or lead to urinary incontinence. Hormone therapy can lead to sexual dysfunction, osteoporosis, anemia, weight gain, fatigue, and other side effects. Current chemotherapeutic agents have side effects ranging from fatigue to hair loss, increased bruising and bleeding, and gastrointestinal effects including loss of appetite, nausea and vomiting, and diarrhea. In some cases, cancers can spread and/or become resistant to available treatments. Thus, new therapies and treatments for controlling prostate tumor growth and invasion are needed.

RNAs that control the progression of cancer are stored and degraded by enzymes found in Processing bodies (P-bodies, PBs). PBs are assemblies of RNA and protein that are not surrounded by a membrane and that form and disappear based on specific cellular queues including, but not limited to, stress conditions such as amino acid starvation during translation or tumor hypoxia. PBs are known sites of localization for the 5′-to-3′ mRNA degradation machinery. It has been discovered that blocking phosphorylation of the PB protein Enhancer of Decapping 3, or EDC3, can control RNA decay and regulate PB formation. Two protein kinases, Pim and AKT, which are known drivers of metastatic PCa, have been shown to phosphorylate EDC3 and inhibit EDC3 from entering P-bodies, thus controlling EDC3's function. It would be highly desirable to develop a small molecule inhibitor that binds the Pim kinase and acts as a substrate competitor (i.e., does not bind to the ATP binding pocket), thereby inhibiting EDC from interacting with Pim. Treatment of PCa with such a molecule would ideally block PB formation, decrease integrin β1 levels, and inhibit PCa tumor growth and metastatic spread.

Pim (provirus integration site for Moloney murine leukemia virus) is a family of serine/threonine kinases that are proto-oncoproteins thought to work in concert with additional proteins to induce tumorigenesis, including, but not limited to, c-Myc. Pim does not appear to require phosphorylation for activation; thus, Pim activity levels are based on cellular concentrations of this protein. Pim kinases may further promote the activity of mTOR (mammalian target of rapamycin), the catalytic subunit of protein complexes mTORC1 and mTORC2. Activity of mTOR or proteins incorporating mTOR may, in turn, enhance the growth of tumor cells, including multiple tumor types e.g., breast tumors, gastrointestinal tumors, and the like. Furthermore, drugs targeting the mTOR pathway have been shown to act against lung tumors and other cancers.

Furthermore, EDC3 phosphorylation and/or other Pim activities have been implicated in the development and spread of other cancers, including, but not limited to, glioma, thyroid cancer, lung cancer, colorectal cancer, head and neck cancer, stomach cancer, liver cancer, pancreatic cancer, renal cancer, urothelial cancer, testis cancer, breast cancer, cervical cancer, endometrial cancer, ovarian cancer, oral squamous cell cancer, hepatocellular carcinoma, bladder cancer, non-small lung cancer, and melanoma. Thus, development of a small molecule inhibitor of Pim kinase may additionally provide treatment options for a variety of other cancers in addition to PCa.

Despite advances in prostate cancer research, there is still a scarcity of compounds that are both potent, efficacious, and selective substrate competitors for Pim kinase and effective in the treatment of prostate cancer and other cancers associated with phosphorylation of EDC3 and elevated mTOR activity. Ideally, the compounds would inhibit protein synthesis and ramp up P-body degradation of mRNAs while exhibiting few systemic side effects when administered to patients. These needs and other needs are satisfied by the present disclosure.

### SUMMARY

In accordance with the purpose(s) of the present disclosure, as embodied and broadly described herein, the disclosure, in one aspect, relates to small molecules useful as inhibitors of Pim and mTOR protein kinases, pharmaceutical compositions comprising the same, and methods of treating cancers associated with phosphorylation of protein Enhancer of Decapping 3 (EDC3) including, but not limited to, prostate cancer, as well as other cancers in which Pim and mTOR protein kinases are implicated, using the same. Also disclosed are combination therapies including at least one disclosed small molecule inhibitor and at least one AKT inhibitor.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-11 show EDC3 is phosphorylated by Pim and AKT and the phosphorylation inhibits PB formation. FIG. 1A: Lysates of various PCa cell line and FIG. 1B: Lysates of PC3LN4 treated with PIMi; Pim447 (3 μM), AKTi; GSK690693 (5 μM) and the combination for 24 hours were analyzed by Western blot with indicated antibodies. FIGS. 1C-1D: Immunofluorescence staining (IF) of EDC3 in PC3-LN4 cells treated for 6 hrs. with or without Pim447 (3 μM) and AKTi; AZD5363 (5 μM) FIG. 1E: PB quantification under different drug treatments, Unpaired t test was performed to calculate statistical significance. FIG. 1F: Western blotting of whole cell lysates (WCL) and purified PBs from GFP (FIG. 1C) and GFP-DCP1a expressing cells with the indicated antibodies FIG. 1G: IF of EDC3 in PC3-LN4 EDC3 S161A cells and FIG. 1H: PC3-LN4 EDC3 S161D treated with Pim447 (3 μM) and AKTi; GSK690693 (5 μM) for 6 hrs. FIG. 1I: IF of EDC3 in RWPE1 cells.

FIGS. 2A-2J show EDC3 expression significantly increases in high grade prostate cancer. Immunohistochemistry for EDC3 (200×); FIG. 2A: normal prostatic glands, FIG. 2B: Gleason score 3, FIG. 2C: high grade Gleason score 4 and FIG. 2D: 5 prostate cancers. Immunohistochemistry for phospho-EDC3 (200×); FIG. 2E: normal prostatic glands, FIG. 2F: Gleason score 3, FIG. 2G: Gleason score 4 and (H) Gleason score 5 prostate cancers. FIGS. 2I-2J: Immunostaining density of EDC3 and p-EDC3 is analyzed among Gleason score group 1 to group 5 (n=6 per group, *p<0.05).

FIGS. 3A-3E show biological impact of EDC3. FIG. 3A: Western blot of CRISPR PC3-LN4 EDC3 ser161 S-D, ser161 S-A mutants and naïve cells, FIG. 3B: Indicated cells were plated (8,000 cell/well in 24 well plate) and imaged every 12 h in the IncuCyte. Graphs was generated with IncuCyte Basic Software graph functions, FIG. 3C: CRISPR PC3-LN4 EDC3 ser161 S-A mutants and naïve cells were injected subcutaneously in a single flank (5×10⁶ cells per animal) and their tumor growth was monitored, FIG. 3D: Migration and FIG. 3E: Invasion assay comparing EDC3 CRISPR cell lines. Unpaired student t-test was performed to calculate statistical significance.

FIGS. 4A-4B show reduction of tumor invasion by unphosphorylated EDC3. FIG. 4A: Tumors in Diaphragm at day 27 after intraperitoneal injection of 5×10⁶ Cells. FIG. 4B: H/E staining in the section.

FIGS. 5A-5C show gene expression profiling in EDC3 S161A mutant cells. FIG. 5A: The heatmap represents the row z-score of log 2 transformed RNA-seq count values from total RNAs of EDC3 S161A and wild type (WT) PC3-LN4 cells. Selected GO term annotations of down regulated cluster is represented on the right side of the heat map. FIG. 5B: Venn diagram showing a comparison between total RNA seq, S-A vs WT (Up_total) or (Down_total) compared to RNAs in PBs UP_P-body (S-A vs WT) or Down_P-body (SA vs WT). FIG. 5C: GMUCT data GO analysis of transcripts that are degraded or decreased in S-A Uncapped RNA compared to S-A total RNA. GO terms are FDR <0.01.

FIGS. 6A-6G show EDC3 SA mutation and dephosphorylation reduced mRNA and protein expression related migration, invasion, and growth. FIG. 6A: Expression of various mRNAs was analyzed by qPCR. FIGS. 6B-6C: Cell lysates of WT and SA cells were subjected to Western blot with the indicated antibodies. FIG. 6D: KLF4 mRNA level was analyzed by qPCR with RNA from PC3-LN4 cells with/without Pimi (Pim447 3 μM) and FIG. 6E: PC3-LN4 Pim-1 Dox inducible cells treated with/without 100 ng/ml of doxycycline. FIG. 6F: Cell lysates of C4-2B and DU145 with/without 3 μM Pim447 were subjected to Western Blot with indicated antibodies. FIG. 6G: smFISH for EDC3 (Green) and KLF (Red) with DAPI staining of the nucleus, yellow representing an overlap of the two.

FIGS. 7A-7I show inhibition of VBT5445 (VBT) on Pim1 and EDC3 binding and phosphorylation. FIG. 7A: In vitro co-precipitation (Co-PPT) assay was performed with recombinant EDC3 and biotinylated Pim1 using Streptavidin beads with increasing concentrations of the Pim inhibitor VBT5445 (VBT). FIG. 7B: Kinase assays were performed with recombinant EDC3 and Pim1 with increasing concentrations of VBT. FIG. 7C: Cell lysates from PC3-LN4 cells cultured with DMSO, 10 μM VBT, 3 μM AZD5363 (AKTi) and the combination for 6 hrs were subjected to western blotting (WB). FIG. 7D: WB of PC3-LN4 cell lysates cultured with Pim447/AZD5363 or VBT/AZD5363 combination treatments. WBs were performed using the indicated antibodies. FIG. 7E: PC3-LN4 cells were seeded into 48 well plate and incubated with either DMSO, VBT, AZD5363 or the combination, for 4.5 days. A real-time imaging system (IncuCyte™) was used to measure cell proliferation using a non-label cell monolayer confluence approach. FIG. 7F-7H: GFP image in PC3 LN4 GFP EDC3 expressing cells treated with PIMi: VBT (5 μM) and VBT (5 μM)+AZD5363 (3 μM) for 6 hrs. FIG. 7I: VBT 5445 in the presence of 500 nM PIM447 or DMSO, SPR was performed on ForteBio Pioneer FE SPR System.

FIG. 8 shows that, in vitro, VBT 1-34 will block the co-immunoprecipitation of EDC3 with Pim, suggesting that it binds to the substrate binding site of Pim. B2 is an inactive analog of VBT 1-34. Consistent with the first observation, VBT 1-34 will inhibit the ability of Pim to phosphorylate EDC3. Data with Pim447, a Pim inhibitor (Novartis Corporation, Basel, Switzerland), is shown as a control.

FIG. 9 GRG 1-34 suppresses Pim kinase activity in prostate cancer cells. Left: LNCaP and LNCaP stably expressed Pim1 cells were treated with DMSO, 5 and 10 μM of GRG 1-34, and 3 μM of Pim447 (P) in 10% FBS medium for 24 h. Right: LNCaP and LNCaP stably expressed Pim1 cells were treated with DMSO and GRG-1-34 (5 M) without FBS medium for 6 h. Lysates (25 μg) were subjected to western blotting with the antibodies for P-IRS1 (S1101), IRS1, P-elF4B (S406), elF4B, P-EDC3 (S161), EDC3, Pim1, and β-Actin.

FIG. 10 shows that treatment of prostate cancer cell lines PC3-LN4 using VBT 1-34 decreases cellular levels of Pim1, c-Myc, and the phosphorylation of the S6 protein, showing inhibition of mTOR protein.

FIG. 11 shows that the addition VBT 1-34 to prostate cancer cells increases the number of P-bodies. Moreover, the addition of the AKT inhibitor AZD 5363 further increased the number of P-bodies that were induced.

FIGS. 12A-12B show the most active analogs disclosed herein.

FIGS. 13A-13B show a comparison of analogs with activity against Panc1 pancreatic cancer cells.

FIG. 14 shows that VBT 1-34 inhibits the growth of pancreatic cancer cells.

FIG. 15 shows that VBT 1-34 inhibits the growth of T-acute lymphoblastic leukemia.

FIG. 16 shows that the inhibition of prostate cancer growth by VBT 1-34 is enhanced by the addition of the AKT/PI3K inhibitors AZD 5363 or BKM 120.

FIG. 17 shows Inhibition of A549 lung cancer growth by GRG-1-34. Cells were plated at 1000 cells per well and 24 h later treated with GRG compounds. After 7 days of growth, tumor cells were fixed with 4% paraformaldehyde, stained with 0.5% crystal violet in methanol and lysed in SDS. The absorbance of six samples treated with each compound or DMSO control was read at 570 nM. The error bars represent the Standard Deviation of these measurements

FIG. 18 shows Inhibition of DU145 prostate cancer growth by GRG1-34. Cell growth was measured as in FIG. 17.

FIG. 19 shows VBT 1-34 inhibits phosphorylation of mTORC substrates. This activity parallels growth inhibition in multiple cell lines. The phosphorylation of three mTORC substrates were inhibited by treatment with VBT 1-34 including AKT on serine 473, S6 protein on serine 235,236, and 4E-BP1 on threonine 37,46.

FIG. 20 shows VBT 1-34 inhibits mTORC even in TSC2 or DEPDC5 KO prostate tumor cells where it would normally be highly activated. This suggests that VBT 1-34 acts downstream from the RAG and RHEB pathways. The activation seen with AKT serine 473 phosphorylation suggests that the disclosed compounds in tumor cells that have a PTEN deletion activate TORC2.

FIG. 21 shows VBT 1-34 can be administered orally and peaks at a level with activity in cell culture. The plasma concentration of VBT 1-34 peaked at 0.5 hr after oral dosing and declined as a function of time. The average maximum plasma concentration (Cₘₐₓ) was 1988 ng/ml following a dose of 62 mg/kg. The half-life (T_{1/2}) of VBT 1-34 was 2.077 hr. It has a large apparent volume of distribution (VZ/F) and apparent systemic clearance (CL/F), likely attributed to low systemic bioavailability (F) after oral dosing. The relative bioavailability between the experiments with the 62 mg/kg dose vs 50 mg/kg dose was 2, indicating a 2× improvement in oral bioavailability with the 62 mg/kg dose/formulation from the 50 mg/kg dose/formation. At 2 ng/ml, the blood concentration of VBT 1-34 reaches 4 μM which in cell culture is sufficient to inhibit tumor growth.

FIG. 22 shows that 6 hour HBSS treatment induces PBs in PC3-LN4 GFP-EDC3 cells. To measure PB numbers, PC3-LN4 PCa cells containing a CRISPR/Cas total knock-out of EDC3 were stably transduced with GFP-EDC3. When localized in PBs, GFP spots could be quantified. PBs were quantified at the University of Colorado Center for Drug Discovery using an Opera Phenix Plus high content screening system (PerkinElmer) Perkin Elmer G3 robotic system after drug treatment. Assays were carried out in quadruplicate and the mean number of PBs per cell and cell area calculated. An example of this assay is shown using Hepes buffered saline (HBSS) to induce PB formation.

FIG. 23 shows inhibition of protein phosphorylation and decrease in protein levels induced by VBT-5445 PC3-LN4 cells were treated with DMSO, VBT5445 (10 μM), AZD5363 (3 μM), or the combination for 24 h. Extracts were subjected to Western blotting with the indicated antibodies.

FIG. 24 shows clustering of GRG target pathways in PC3-LN4 and PANC1 cells. Tumor cells were treated with or without GRG-1-34 (5 μM) for 24 h and lysates were subjected to RPPA. The results were clustered in the form of heat map created using the Heatmapper web tool. The analysis was completed with the clustering method of Complete Linkage and the distance measurement method of Kendall's Tau.

FIG. 25 shows Induction of Acetyl CoA carboxylase phosphorylation by GRG-1-35. LNCaP cells were treated in a 6 well plate with GRG-1-35 for the indicated times. Cells were scrapped and protein extracts subjected to SDS-PAGE and Western blotted with the antibodies shown.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The protein level of drivers of prostate cancer (PCa) growth, invasion, and metastasis are controlled in large part by the balance in transcription, translation, and mRNA decay. To inhibit cancer growth, translation has been successfully targeted in patients. However, the mRNA decay pathway, the other half of this control mechanism, has not been a focus of anticancer therapy development. The present disclosure demonstrates that it is possible to alter sites of RNA storage and degradation, called processing bodies (P-bodies, PBs), to inhibit PCa cell growth, motility and invasion in both tissue culture and animal models. In one aspect, this represents a novel pathway that can be targeted to develop unique approaches to anticancer therapy.

In one aspect, protein kinases activated in PCa phosphorylate and control the activity of Enhancer of Decapping 3 (EDC3), an important P-body protein. In a further aspect, it has been discovered that protein kinases phosphorylate EDC3 on serine 161 (S161) altering this protein's localization and preventing the formation of PBs1. Further in this aspect, the Pim and AKT protein kinases, whose activity is highly elevated in PCa, phosphorylate this site. In one aspect, these protein kinases play a central role in controlling PCa growth and invasion. In another aspect, moderate to strong cytoplasmic staining of Pim1 is reported in 68% tumor patients with a Gleason score of seven or higher. In a still further aspect, nuclear Pim has been associated with a higher risk of PSA recurrence and with perineural invasion of the prostate gland, indicating that Pim plays a central role in controlling PCa metastasis. In one aspect, AKT activity is elevated in PCa secondary to chromosomal deletion and mutation of the PTEN in 50-80% of PCa patients. In an aspect, levels of p-AKT and its activated form are associated with higher Gleason grade tumors and poor prognosis and, further, predict disease recurrence.

In one aspect, the EDC3 protein normally functions to increase decapping by binding to mRNAs and proteins in the decapping complex (Dcp1/Dcp2), and, in addition, regulating mRNA storage in PBs. In a further aspect, alterations in mRNA decapping efficiency radically change mRNA stability affecting the production of specific proteins driving the growth and spread of PCa cells. In one aspect, the ability to control the mRNA and protein composition of PBs can be important to regulating protein production in PCa. In an aspect, P-bodies (PBs) are membrane-less condensates that contain core 5′-3′ mRNA decay proteins, including the decapping complex, decapping enhancers/translation repressors (DDX6, EDC3, EDC4), and the 5′-3′ exonuclease XRN1 as well as Poly-A binding proteins and miRNAs. In a further aspect, cellular stresses, including nutrient and growth factor starvation, cause these proteins to assemble in PBs. In another aspect, the observation that phosphorylation of EDC3 at Ser 161 in PCa cells prevents PB formation suggests that this modification plays a key role in regulating mRNA fate. In still another aspect, supporting this hypothesis is the observation that EDC3 along with Dcp1 and Dcp2 increases the rate of RNA decapping in condensates in vitro.

In one aspect, PB formation is driven in large part by proteins that self-interact and/or that harbor intrinsically disordered regions, such as, for example, EDC3. In a further aspect, control of PB formation can be explained by strong protein-protein, protein-RNA, and RNA-RNA interactions that promote liquid-liquid phase separation (LLPS) and condensate formation. In a still further aspect, LLPS is widely utilized compartmentalization mechanism in cells that regulates multiple cellular processes including transcription, translation, RNA storage and degradation. In one aspect, S161 lies in a conserved disorder region of EDC3, which in S. pombe facilitates EDC3-RNA interactions, and aids assembly of PBs by promoting EDC3 oligomerization and binding to Dcp1/Dcp2 complexes. Further in this aspect, S. pombe EDC3 functions in condensates by removing the autoinhibition of the Dcp1/Dcp2 complex and markedly enhancing 5′-decapping and mRNA degradation. In a further aspect, however, the role of the human EDC3 IDR on the above processes, and S161 phosphorylation, is not known. In one aspect, over 125 proteins have been identified in PBs, many of which may interact with EDC3 indirectly, and they recruit over 6,000 species of RNAs to these assemblies. In a further aspect, it is believed that IDR phosphorylation may affect EDC3 structure or the scaffolding function of EDC3, blocking its interaction with PB proteins, and PB assembly. In an alternative aspect, EDC3 phosphorylation could alter its affinity for mRNAs by inhibiting self-dimerization, thus affecting mRNA targeting to PBs. Finally, EDC3 S161 phosphorylation may regulate the rate of decapping induced by Dcp1/Dcp2 in PBs. In one aspect, these possibilities are not mutually exclusive.

In one aspect, mutation of the EDC3 phosphorylation site markedly inhibits prostate tumor growth, invasion, and migration. In a further aspect, in cell culture and animal models, PCa cells expressing the phospho-mutant S161A of EDC3 show markedly decreased cell growth and motility and fail to invade smooth muscle. In a still further aspect, RNA seq. analysis of mutant and wild type cells followed by clustering and Gene Ontology analysis demonstrates that there is a marked decrease in pathways that encode genes for cell migration, cell-cell adhesion, cell proliferation, and cytokine mediated signaling. In another aspect, PCa cells expressing EDC3 S161A contain markedly decreased protein and mRNA levels of integrin β1 and α6 as well as multiple other proteins associated with these pathways. In yet another aspect, PCa cells expressing integrin α6 and β1, α6β1, metastasize to bone invading along nerves which contain laminin pointing to the importance of regulation of these genes. In one aspect, it is believed that controlling RNA decay has a significant impact in alteration of EDC3 phosphorylation-driven regulation of integrin levels.

In one aspect, disclosed herein is a new small molecule tool compound that is a substrate competitor of EDC3 and prevents binding to Pim. In another aspect, unlike most other kinase inhibitors this tool compound does not bind to the ATP binding pocket or increase the level of Pim in cells when administered. In a further aspect, this small molecule, VBT5445, can inhibit EDC3 binding to Pim and can be used as a tool to investigate the importance of this interaction. In still another aspect, the development of this small molecule points to a new approach to inhibit Pim-EDC3 interaction. Also disclosed herein are additional small molecule inhibitors in the same class.

In one aspect, this compound not only inhibits Pim but is also capable of inhibiting the mTOR protein kinase. In a further aspect, this is a unique combination inhibition for a single molecule. In one aspect, and without wishing to be bound by theory, by inhibiting mTOR, mRNA that is not being translated can either be stored or degraded. In a further aspect, this compound's inhibition of Pim can enhance EDC3 function and thus lead to the degradation of specific RNAs in P-bodies. In one aspect, mTOR inhibition alone is known to inhibit tumor growth by regulating protein production and increasing the level of reactive oxygen species in tumor cells.

In another aspect, triple-negative breast cancer displays elevated MYC signaling along with PIM expression. In a further aspect, small molecule PIM kinase inhibitors such as those disclosed herein may be particularly useful in treating breast cancer. In a further aspect, when the PI3K/AKT pathway is inhibited in breast cancer, Pim is increased so that, in any breast cancer treated with the disclosed agents, this class of inhibitors may have activity.

Disclosed herein are compounds having a structure according to Formula I:

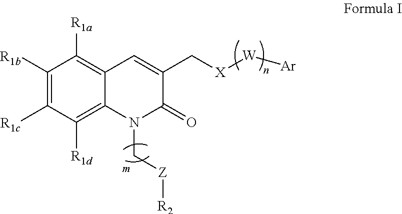

wherein each of R₁ₐ-R_{1d} is independently selected from hydrogen, halogen, or

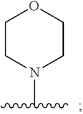

wherein m is from 1 to 6;
wherein Z is O, NH, NR₆, or S;
wherein R₂ is substituted or unsubstituted aryl or heteroaryl;
wherein R₆ is hydrogen, substituted or unsubstituted C₁-C₁₀ linear or branched alkyl or substituted or unsubstituted alkyne;
wherein X is NR₃, O, or S;
   wherein, when X is NR₃, R₃ is hydrogen, substituted or unsubstituted C₁-C₁₀ linear or branched alkyl, or substituted or unsubstituted alkyne;
wherein each occurrence of W is CH₂ or C(═O);
wherein n is from 0 to 4;
and wherein Ar is a substituted or unsubstituted C₁-C₁₀ aryl or heteroaryl group.

In another aspect, R₁ₐ, R_{1b}, and Rid are hydrogen and R_{1c} is halogen. In some aspects, R_{1c} is chlorine. In still another aspect, R₁ₐ, R_{1b}, and R_{1d} are hydrogen and R_{1c} is

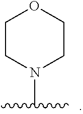 [SMILES: *N1CCOCC1]

In one aspect, m is 2. In another aspect, R₂ is phenyl or

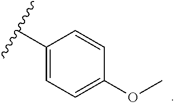 [SMILES: *c1ccc(OC)cc1]

In one aspect, Z is S and R₂ is phenyl. In another aspect, Z is O and R₂ is phenyl. In still another aspect, Z is O and R₂ is

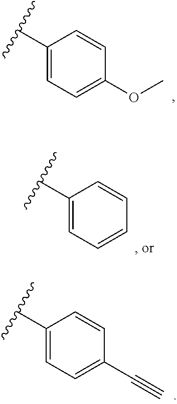

In one aspect, X is O. In another aspect, X is NR₃ and R₃ is H. In one aspect, X is NR₃ and R₃ is methyl. In yet another aspect, X can be NR₃ and R₃ can be

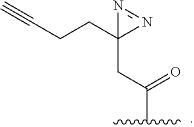 [SMILES: *C(=O)CC1(CCC#C)N=N1]

In any of these aspects, n can be 0 or 1. In one aspect, n is 1 and W is CH₂. In another aspect, n is 1 and W is C(═O).

In one aspect, Ar can be

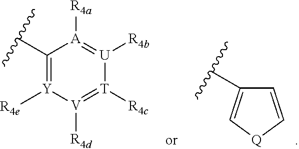

In another aspect, Y can be N and R₄ₑ can be absent, or V can be N and Rad can be absent, or T can be N and R_{4c} can be absent, or U can be N and R_{4b} can be absent, or A can be N and R₄ₐ can be absent. In an alternative aspect, A, T, U, V, Y, or any combination thereof can be C. and R₄ₐ-R₄ₑ, if present, can independently be selected from hydrogen, halogen, alkoxy, hydroxy, or substituted or unsubstituted C₁-C₄ alkyl. In some aspects, R_{4b} and R_{4c} together form a fused 5- or 6-membered aryl or heteroaryl ring.

In still another aspect, Q can be O, S, or NR₅, wherein R₅ is hydrogen or C₁-C₄ alkyl.

Non-limiting examples of Ar include:

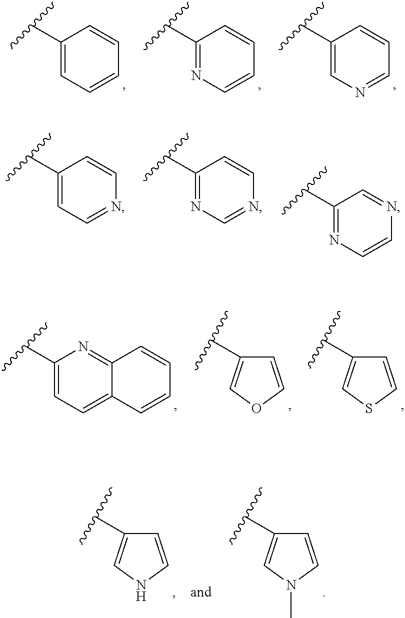

In any of these aspects, Y can be CH and each of R₄ₐ-R_{4d} is hydrogen, or Y can be N. In one aspect, each of R₄ₐ, R_{4c}, and Rad is hydrogen and R_{4b} is selected from bromo, chloro, fluoro, trifluoromethyl, methyl, or methoxy. In another aspect, each of R₄ₐ, R_{4b}, and R_{4d} is hydrogen and R_{4c} is selected from chloro, trifluoromethyl, or methoxy. In one aspect, each of R_{4b}, R_{4c}, and R_{4d} is hydrogen and R₄ₐ is selected from bromo or fluoro. In one aspect, each of R₄ₐ, R_{4b}, and R_{4c} is hydrogen and R_{4d} is selected from bromo or fluoro. In another aspect, each of R₄ₐ, R_{4b}, and R_{4c} is hydrogen and Rad is selected from methyl or hydroxy.

In one aspect, the compound can be selected from

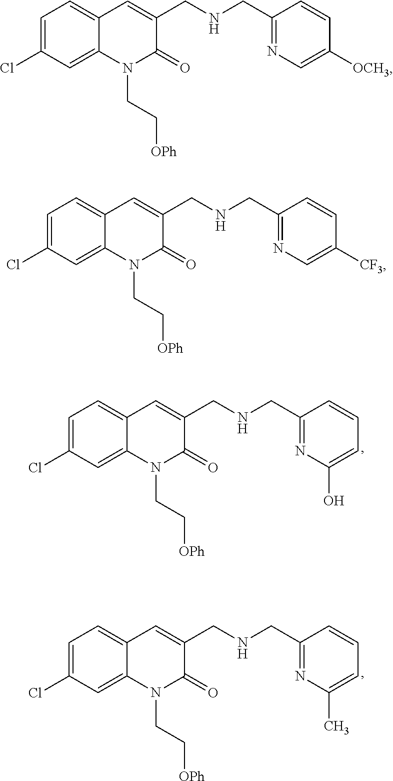 [SMILES: COc1ccc(CNCc2cc3ccc(Cl)cc3n(CCOc3ccccc3)c2=O)nc1 ; O=c1c(CNCc2ccc(C(F)(F)F)cn2)cc2ccc(Cl)cc2n1CCOc1ccccc1 ; O=c1c(CNCc2cccc(O)n2)cc2ccc(Cl)cc2n1CCOc1ccccc1 ; Cc1cccc(CNCc2cc3ccc(Cl)cc3n(CCOc3ccccc3)c2=O)n1]

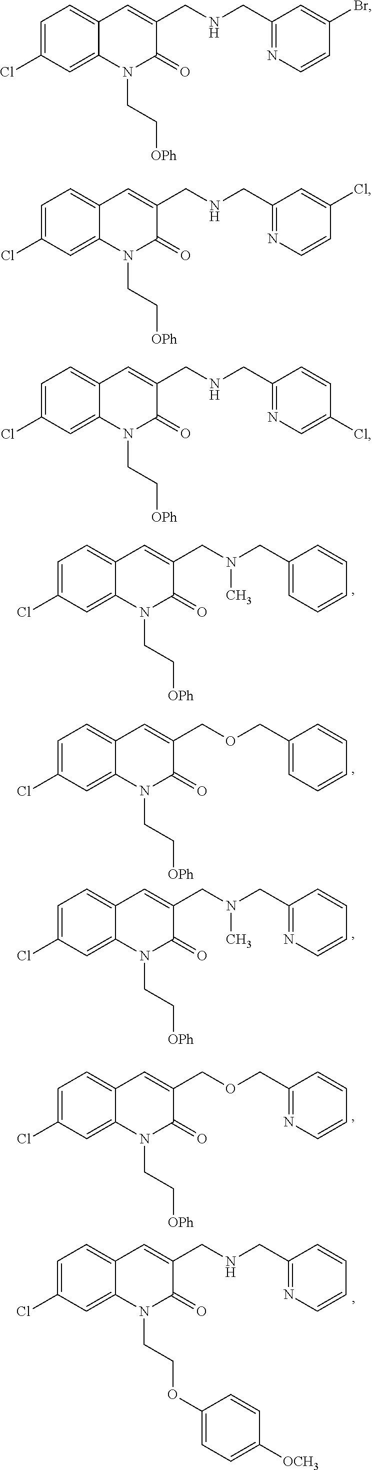

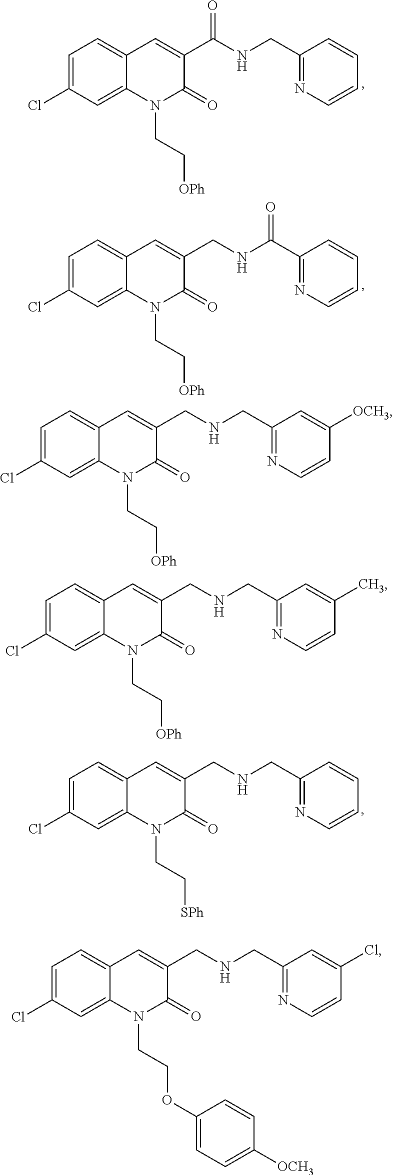

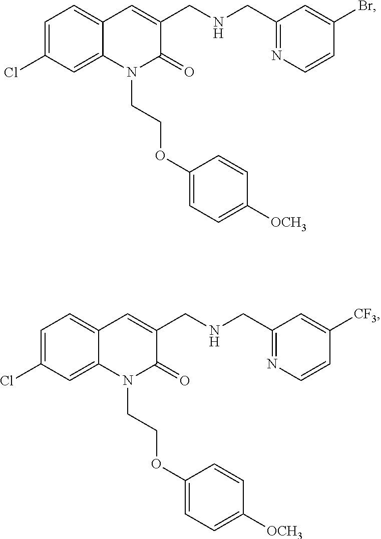 [SMILES: COc1ccc(OCCn2c(=O)c(CNCc3cc(Br)ccn3)cc3ccc(Cl)cc32)cc1 ; COc1ccc(OCCn2c(=O)c(CNCc3cc(C(F)(F)F)ccn3)cc3ccc(Cl)cc32)cc1]

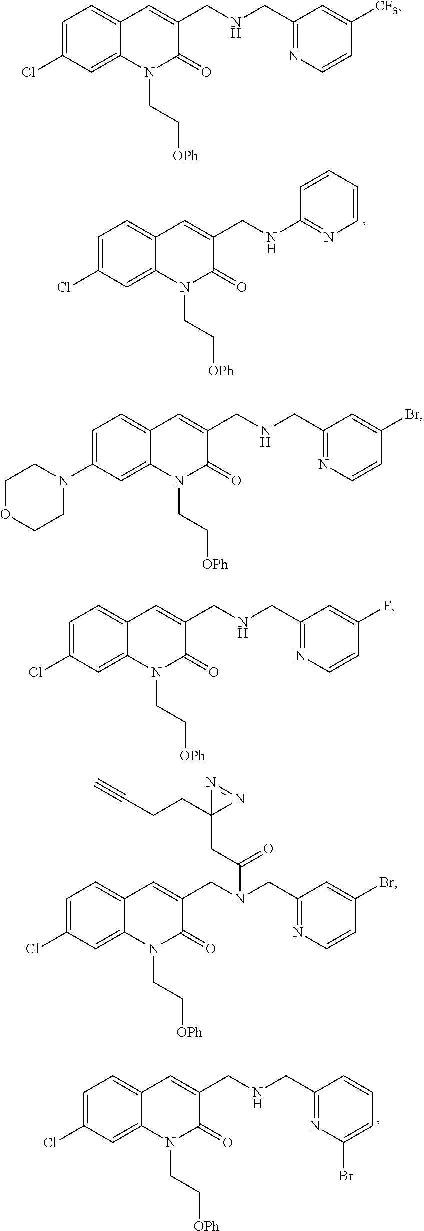

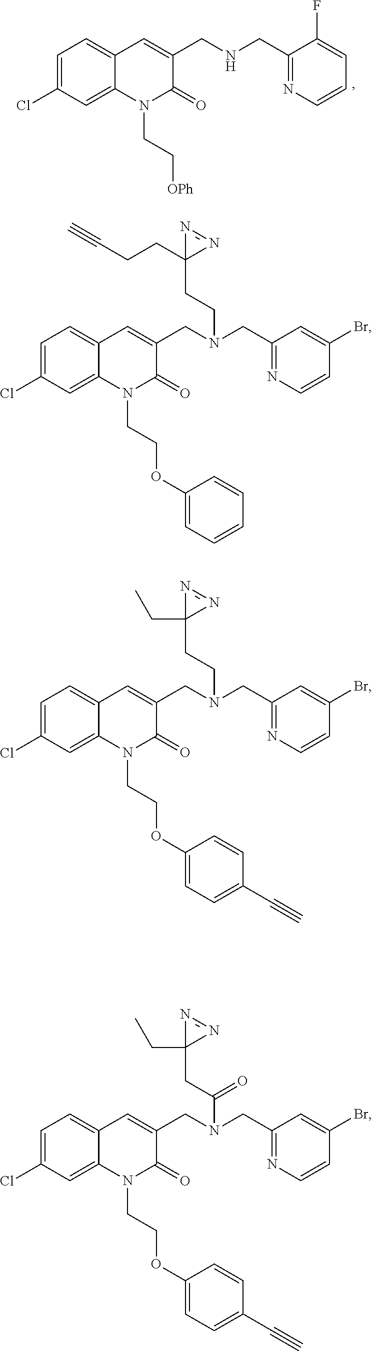

or any combination thereof.

In any of these aspects, the compound can be an allosteric Pim kinase inhibitor, an mTOR inhibitor, or both.

Also disclosed herein are pharmaceutical compositions including at least one disclosed compound or a pharmaceutically acceptable salt thereof. In another aspect, the pharmaceutical compositions can include at least one excipient. In still another aspect, the pharmaceutical composition can include at least one AKT inhibitor such as, for example, Akti-1/2, API-1, API-2, AT 7867, AZD 5363, 10-DEBC hydrochloride, FPA 124, GSK 690693, Perifosine, PHT 427, SC 66, KP372-1, AKTide-2T TFA, AKTide-2T, SC79, Honokiol, TD52, TASP0415914, ACT001, Artemisinin, Recilisib, Guggulsterone, Scutellarin, Triciribine, α-linolenic acid, miltefosine, deguelin, LM22B-10, 1,3-dicaffeoylquinic acid, pachymic acid, cenisertib, sophocarpine, esculetin, borussertib, Paris saponin VII, amicolide D, N-oleoyl glycine, CHPG, hematein, loureirin A, phellodendrine, PHT-427, deltonin, crosstide, N-feruloyloctopamine, glaucocalyxin A, CAY 10404, polygalasaponin F, hederacolchiside A1, rotundic acid, K-80003, sophocarpine monohydrate, MPTOE028, kazonil B, batatasin III, 8-aminoadenosine, sennidin B, sennidin A, or any combination thereof.

In another aspect, the pharmaceutical composition can include at least one additional Pim kinase inhibitor. In another aspect, the at least one additional Pim kinase inhibitor can be SGI-1776, NVP-LGB321, a Pim-specific siRNA, or any combination thereof. In still another aspect, the pharmaceutical composition can further include at least one mTOR inhibitor. In a further aspect, the at least one mTOR inhibitor can be selected from everolimus, deferolimus, gefitinib, temsirolimus, torin-1, torin-2, vistusertib, PP242, ridaforolimus, umirolimus, zotarolimus, rapamycin, a rapamycin derivative, or any combination thereof.

Further disclosed herein is a method for treating at least one disease or disorder, the method including administering a disclosed compound or pharmaceutical composition to a subject. In another aspect, the disease can be glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, oral squamous cell cancer, hepatocellular carcinoma, bladder cancer, non-small lung cancer, melanoma, or any combination thereof.

In an alternative aspect, the disorder can be a metabolic disorder such as, for example, hypercholesterolemia, type 1 diabetes, type 2 diabetes, gestational diabetes, metabolic syndrome, obesity, or any combination thereof. In one aspect, accumulation of fat in animal tissues is associated with insulin resistance. In a further aspect, the disclosed compounds can, through their activity, inhibit acetyl CoA carboxylase by phosphorylation, and thus decrease the production of long chain fatty acids. In a still further aspect, decreases in long chain fatty acid production will increase insulin sensitivity. In one aspect, activation of the AMPK kinase by these compounds can enhance energy production upon exercise. In still another aspect, the genes ACC1 and ACC2 (acetyl coenzyme A carboxylases 1 and 2, respectively) are implicated in fatty acid storage and metabolism. Further in this aspect, regulation of ACC1 and ACC2 is complex and involves both diet and hormones. Several different protein kinases phosphorylate ACC1 and ACC2 and further reduce the activity of these enzyme isoforms. In one aspect, low levels of ACC2 or inactive ACC2 can lead to improved insulin signaling in animal tissues. When levels of fatty acyl-CoA are high, a protein kinase cascade is initiated, acting on insulin receptor substrates, which may in turn down regulate other kinases such as, for example, PI3 (phosphoinositol 3) kinase and AKT. Thus, in one aspect, the disclosed compounds and pharmaceutical compositions, through their regulation of protein kinases, and thus phosphorylation, may have therapeutic application to the regulation of fatty acid metabolism and, hence, treatment of various metabolic disorders.

In one aspect, the compound or pharmaceutical composition can be administered to the subject orally, by inhalation, parenterally, intravenously, mucosally, or any combination thereof. In a further aspect, the subject can be a mammal such as, for example, a cat, dog, rat, mouse, guinea pig, hamster, rabbit, horse, cattle, swine, sheep, goat, human, or another primate.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, references to “a Pim kinase inhibitor,” “a cancer,” or “an excipient,” include, but are not limited to, mixtures or combinations of two or more such Pim kinase inhibitors, cancers, or excipients, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of an excipient refers to an amount that is sufficient to achieve the desired improvement in the property modulated by the formulation component, e.g. achieving the desired texture or consistency. The specific level in terms of wt % in a composition required as an effective amount will depend upon a variety of factors including the amount and type of Pim kinase inhibitor, type of cancer to be treated, stage of cancer to be treated, method of administration, body weight of the subject undergoing treatment, and the like.

As used herein, the terms “optional” or “optionally” means that the subsequently described event or circumstance can or cannot occur, and that the description includes instances where said event or circumstance occurs and instances where it does not.

Unless otherwise specified, pressures referred to herein are based on atmospheric pressure (i.e. one atmosphere).

### Chemical Compounds and Residues

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e., each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized TT electrons above and below the plane of the molecule, where the TT clouds contain (4n+2) TT electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O)ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C₆ heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S) R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘}) R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘)}₂; or —(C₁₋₄ straight or branched alkylene) C(O)O—N(R_{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene) C(O)OR^{●}, or —SSR^{●} wherein each R^{●} is unsubstituted or where preceded by “halo” is substituted only with one or more halogens, and is independently selected from C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR*, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR*, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

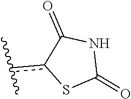

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and(S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or(S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine, and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an a-hydrogen can exist in an equilibrium of the keto form and the enol form.

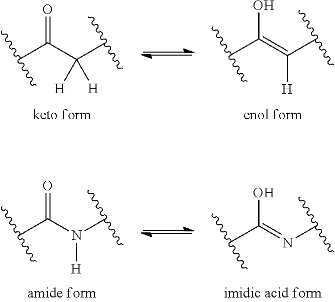 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

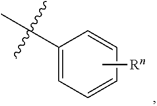

which is understood to be equivalent to a formula:

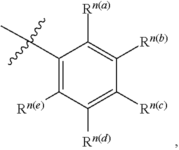

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance Rn (a) is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### Pharmaceutical Compositions and Methods of Use Thereof

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, troubleshooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof, such as prostate tumors and/or growth, invasion, or progression of the same. The effect can be therapeutic in terms of a partial or complete cure of a disease, condition, symptom, or adverse effect attributed to the disease, disorder, or condition. The term “treatment” as used herein can include any treatment of prostate tumors and/or their growth or invasion of other tissues in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder, or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

It is understood, that unless otherwise specified, temperatures referred to herein are based on atmospheric pressure (i.e. one atmosphere).

Described herein are small molecule Pim kinase inhibitors that have therapeutic or clinical utility. Also described herein are methods of administering the small molecules to a subject in need thereof. In some aspects, the subject can have a cancer such as, for example, prostate cancer Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

In various aspects, it is contemplated herein that the disclosed compounds further comprise their biosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, Sep. 10, 2011, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape, and electron density. Preferred bioisosteres of esters, amides or carboxylic acids are compounds containing two sites for hydrogen bond acceptance. In one embodiment, the ester, amide, or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid, and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quatemized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides, and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate, or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially, and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets, or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs, and solutions; while carriers such as starches, sugars, microcrystalline cellulose, diluents, granulating agents, lubricants, binders, disintegrating agents, and the like can be used to form oral solid preparations such as powders, capsules, and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate, and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate, and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N, N-diethyl acetamide, N, N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl) imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants, and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g., 2-hydroxypropyl-β-cyclodextrin or sulfobutyl-β-cyclodextrin. Also, co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long-acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus, and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier, and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic, or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin, and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require modulation of EDC3 activity, an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for modulating EDC3 activity (e.g., treatment of one or more cancers or other disorders associated with EDC3 dysfunction) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, a polymorph thereof, and a small molecule Pim kinase inhibitor. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional anticancer therapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

### Aspects

The present disclosure can be described in accordance with the following numbered Aspects, which should not be confused with the claims.

Aspect 1. A compound having a structure of Formula I:

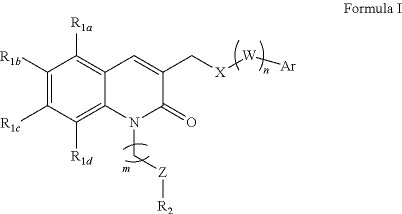

wherein each of R₁ₐ-R_{1d} is independently selected from hydrogen, halogen, or

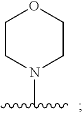

wherein m is from 1 to 6;
wherein Z is O, NH, NR₆, or S;
wherein R₂ is substituted or unsubstituted aryl or heteroaryl;
wherein R₆ is hydrogen, substituted or unsubstituted C₁-C₁₀ linear or branched alkyl, or substituted or unsubstituted alkyne;
wherein X is NR₃, O, or S;
   wherein, when X is NR₃, R₃ is hydrogen, substituted or unsubstituted C₁-C₁₀ linear or branched alkyl, or substituted or unsubstituted alkyne;

wherein each occurrence of W is CH₂ or C(═O);

wherein n is from 0 to 4;

and wherein Ar is a substituted or unsubstituted C₁-C₁₀ aryl or heteroaryl group.

Aspect 2. The compound of aspect 1, wherein R₁ₐ, R_{1b}, and R_{1d} are hydrogen and R_{1c} is halogen.

Aspect 3. The compound of aspect 2, wherein R_{1c} is chlorine.

Aspect 4. The compound of aspect 1, wherein R₁ₐ, R_{1b}, and R₁ₐ are hydrogen and R_{1c} is

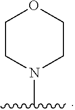 [SMILES: *N1CCOCC1]

Aspect 5. The compound of any one of aspects 1-4, wherein m is 2.

Aspect 6. The compound of any one of aspects 1-5, wherein R₂ is

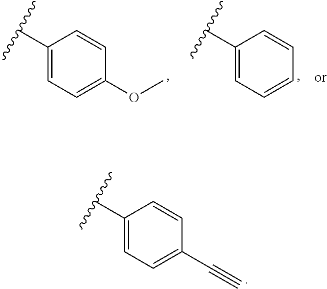

Aspect 7. The compound of any one of aspects 1-6, wherein Z is S and R₂ is phenyl.

Aspect 8. The compound of any one of aspects 1-6, wherein Z is O and R₂ is phenyl.

Aspect 9. The compound of any one of aspects 1-6, wherein Z is O and R₂ is

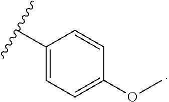 [SMILES: *c1ccc(OC)cc1]

Aspect 10. The compound of any one of aspects 1-9, wherein X is O.

Aspect 11. The compound of any one of aspects 1-9, wherein X is NR₃ and R₃ is H.

Aspect 12. The compound of any one of aspects 1-9, wherein X is NR₃ and R₃ is methyl.

Aspect 13. The compound of any one of aspects 1-9, wherein X is NR₃ and R₃ is

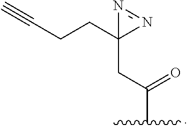 [SMILES: *C(=O)CC1(CCC#C)N=N1]

Aspect 14. The compound of any one of aspects 1-13, wherein n is 0 or 1.

Aspect 15. The compound of aspect 14, wherein n is 1 and W is CH₂.

Aspect 16. The compound of aspect 14, wherein n is 1 and W is C(═O).

Aspect 17. The compound of any one of aspects 1-16, wherein Ar is

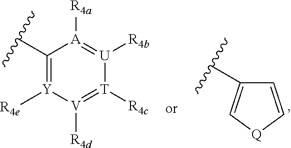

wherein A is C or N, wherein when A is N, R₄ₐ is absent;
wherein U is C or N, wherein when U is N, R_{4b} is absent;
wherein T is C or N, wherein when T is N, R_{4c} is absent;
wherein V is C or N, wherein when V is N, R_{4d} is absent;
wherein Y is C or N, wherein when Y is N, R₄ₑ is absent;
wherein R₄ₐ-R₄ₑ, if present, are independently selected from hydrogen, halogen, alkoxy, hydroxy, or substituted or unsubstituted C₁-C₄ alkyl, or wherein R_{4b} and R_{4c} together form a 5- or 6-membered aryl or heteroaryl ring;
wherein Q is selected from O, S, or NR₅, wherein R₅ comprises hydrogen or C₁-C₄ alkyl.

Aspect 18. The compound of any one of aspects 1-17, wherein Ar is

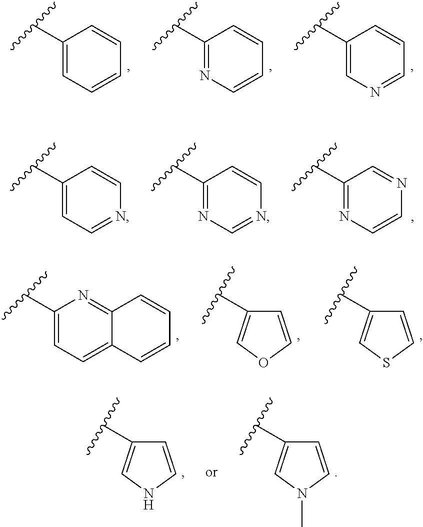

Aspect 19. The compound of aspect 17 or 18, wherein Y is CH and each of R₄ₐ-R_{4d} is hydrogen.

Aspect 20. The compound of aspect 17 or 18, wherein Y is N.

Aspect 21. The compound of aspect 17, wherein each of R₄ₐ-R_{4d} is hydrogen.

Aspect 22. The compound of aspect 17, wherein each of R₄ₐ, R_{4c}, and R_{4d} is hydrogen and R_{4b} is selected from bromo, chloro, fluoro, trifluoromethyl, methyl, or methoxy.

Aspect 23. The compound of aspect 17, wherein each of R₄ₐ, R_{4b}, and Rad is hydrogen and R_{4c} is selected from chloro, trifluoromethyl, or methoxy.

Aspect 24. The compound of aspect 17, wherein each of R_{4b}, R_{4c}, and Rad is hydrogen and R₄ₐ is selected from bromo or fluoro.

Aspect 25. The compound of aspect 17, wherein each of R₄ₐ, R_{4b}, and R_{4c} is hydrogen and R_{4d} is selected from bromo or fluoro.

Aspect 26. The compound of aspect 17, wherein each of R₄ₐ, R_{4b}, and R_{4c} is hydrogen and R_{4d} is selected from methyl or hydroxy.

Aspect 27. The compound of any one of aspects 1-26, wherein the compound is

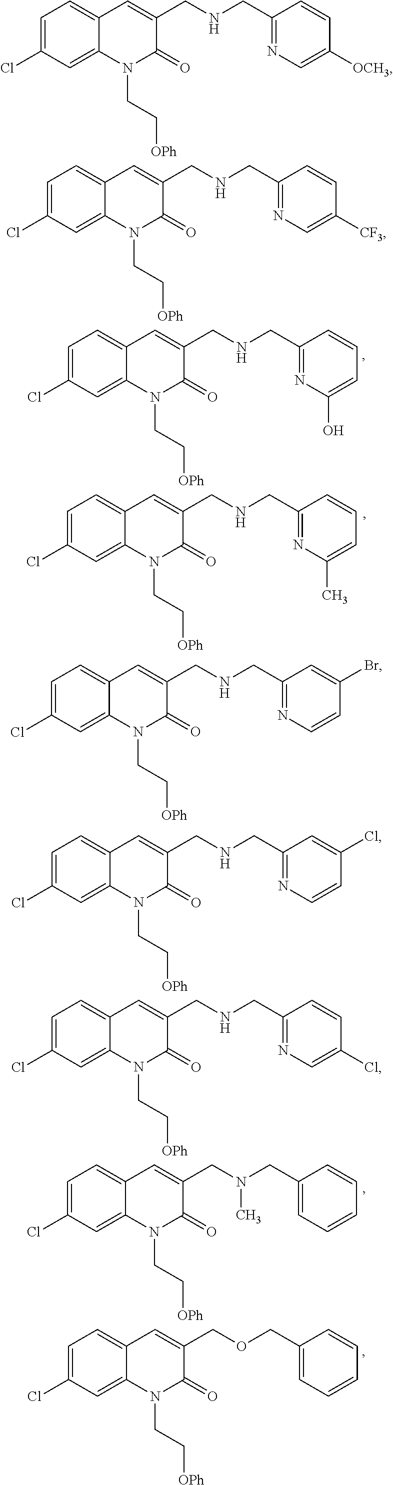

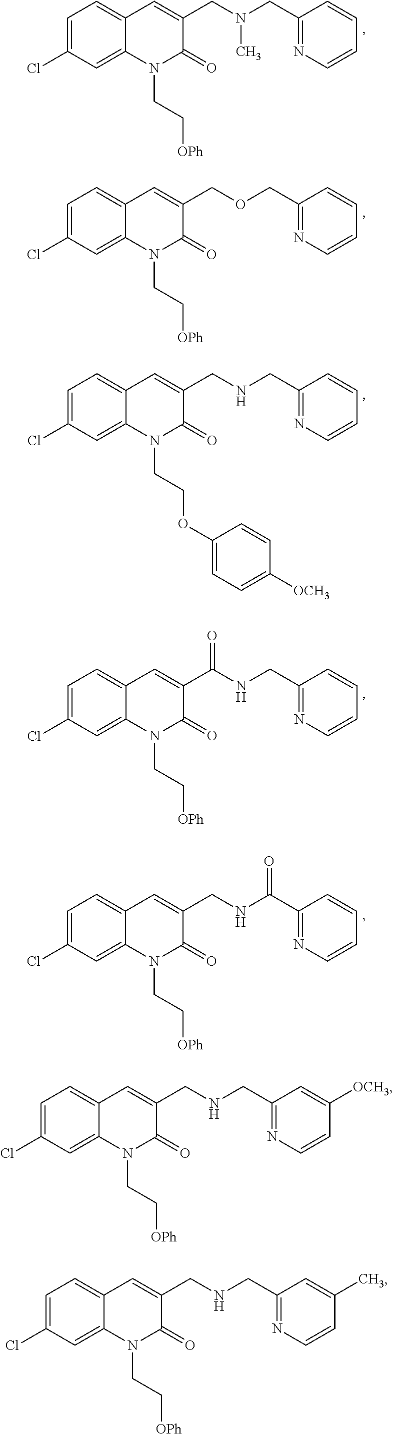

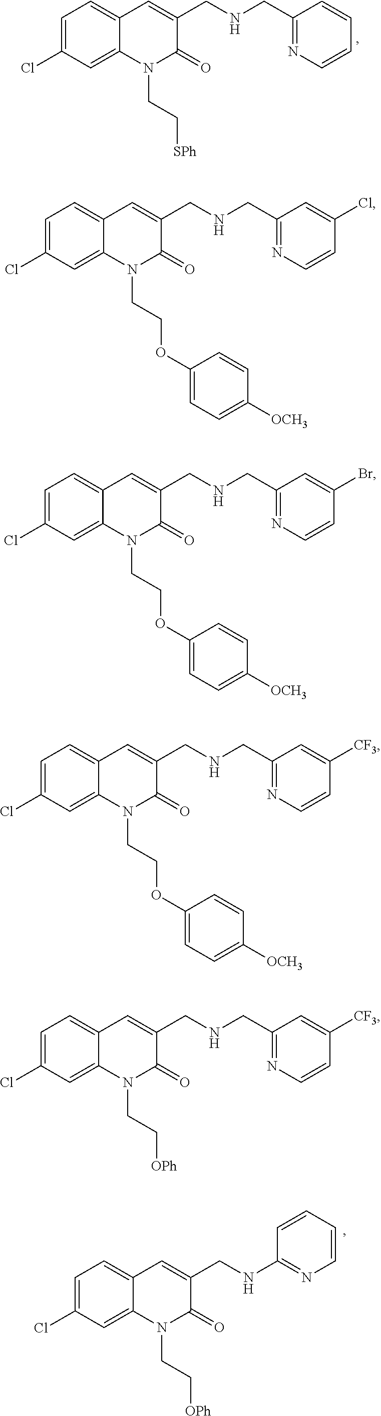

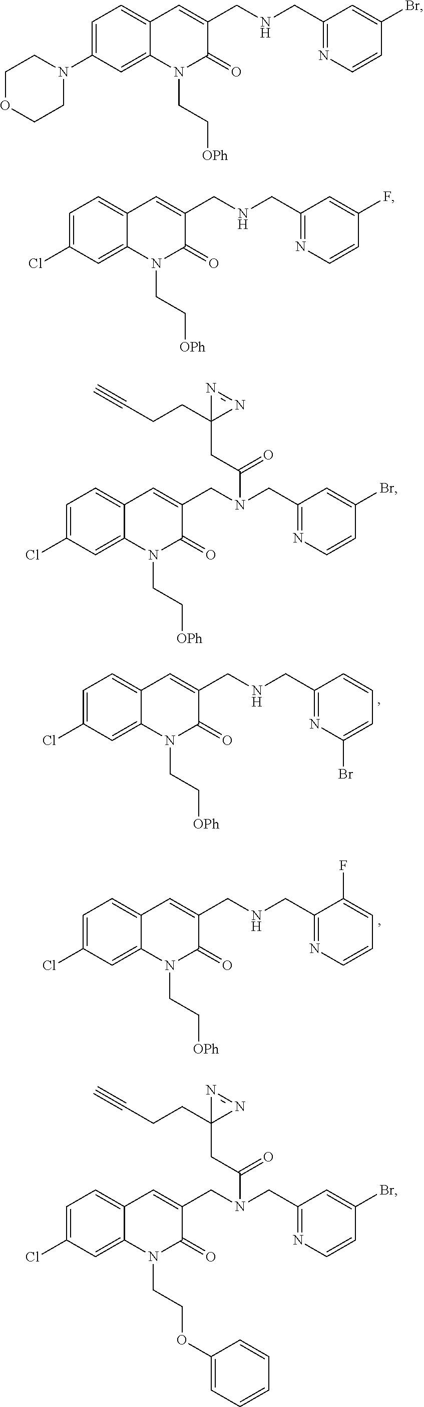

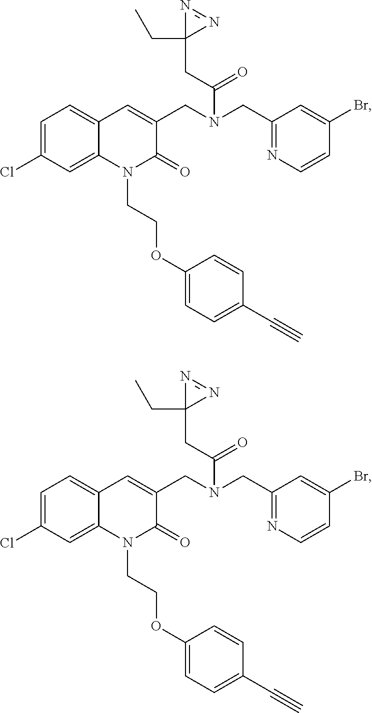 [SMILES: C#Cc1ccc(OCCn2c(=O)c(CN(Cc3cc(Br)ccn3)C(=O)CC3(CC)N=N3)cc3ccc(Cl)cc32)cc1 ; C#Cc1ccc(OCCn2c(=O)c(CN(Cc3cc(Br)ccn3)C(=O)CC3(CC)N=N3)cc3ccc(Cl)cc32)cc1]

or any combination thereof.

Aspect 28. The compound of any one of aspects 1-27, wherein the compound is an allosteric Pim kinase inhibitor.

Aspect 29. The compound of any one of aspects 1-28, wherein the compound is an mTOR inhibitor.

Aspect 30. A pharmaceutical composition comprising the compound of any one of aspects 1-29 or a pharmaceutically acceptable salt thereof.

Aspect 31. The pharmaceutical composition of aspect 30, further comprising at least one excipient.

Aspect 32. The pharmaceutical composition of aspect 30 or 31, further comprising at least one AKT inhibitor.

Aspect 33. The pharmaceutical composition of aspect 32, wherein the at least one AKT inhibitor comprises Akti-1/2, API-1, API-2, AT 7867, AZD 5363, 10-DEBC hydrochloride, FPA 124, GSK 690693, Perifosine, PHT 427, SC 66, KP372-1, AKTide-2T TFA, AKTide-2T, SC79, Honokiol, TD52, TASP0415914, ACT001, Artemisinin, Recilisib, Guggulsterone, Scutellarin, Triciribine, a-linolenic acid, miltefosine, deguelin, LM22B-10, 1,3-dicaffeoylquinic acid, pachymic acid, cenisertib, sophocarpine, esculetin, borussertib, Paris saponin VII, amicolide D, N-oleoyl glycine, CHPG, hematein, loureirin A, phellodendrine, PHT-427, deltonin, crosstide, N-feruloyloctopamine, glaucocalyxin A, CAY 10404, polygalasaponin F, hederacolchiside A1, rotundic acid, K-80003, sophocarpine monohydrate, MPTOE028, kazonil B, batatasin III, 8-aminoadenosine, sennidin B, sennidin A, or any combination thereof.

Aspect 34. The pharmaceutical composition of any one of aspects 30-33, further comprising at least one additional Pim kinase inhibitor.

Aspect 35. The pharmaceutical composition of aspect 34, wherein the at least one additional Pim kinase inhibitor comprises SGI-1776, NVP-LGB321, a Pim-specific siRNA, or any combination thereof.

Aspect 36. The pharmaceutical composition of any one of aspects 30-35, further comprising at least one mTOR inhibitor.

Aspect 37. The pharmaceutical composition of aspect 36, wherein the at least one mTOR inhibitor comprises everolimus, deferolimus, gefitinib, temsirolimus, torin-1, torin-2, vistusertib, PP242, ridaforolimus, umirolimus, zotarolimus, rapamycin, a rapamycin derivative, or any combination thereof.

Aspect 38. A method for treating at least one disease or disorder, the method comprising administering the compound of any one of aspects 1-29 or the pharmaceutical composition of any one of aspects 30-37 to a subject.

Aspect 39. The method of aspect 38, wherein the disease comprises glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, oral squamous cell cancer, hepatocellular carcinoma, bladder cancer, non-small lung cancer, melanoma, or any combination thereof.

Aspect 40. The method of aspect 38 or 39, wherein the disease is prostate cancer.

Aspect 41. The method of aspect 38, wherein the disorder comprises a metabolic disorder.

Aspect 42. The method of aspect 41, wherein the metabolic disorder comprises hypercholesterolemia, type 1 diabetes, type 2 diabetes, gestational diabetes, metabolic syndrome, obesity, or any combination thereof.

Aspect 43. The method of any one of aspects 38-42, wherein the compound or pharmaceutical composition is administered to the subject orally, by inhalation, parenterally, intravenously, mucosally, or any combination thereof.

Aspect 44. The method of any one of aspects 38-43, wherein the subject is a mammal.

Aspect 45. The method of aspect 44, wherein the mammal is a cat, dog, rat, mouse, guinea pig, hamster, rabbit, horse, cattle, swine, sheep, goat, human, or another primate.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Phosphorylation of EDC3 is Blocked by Pim/AKT Kinase Inhibitors

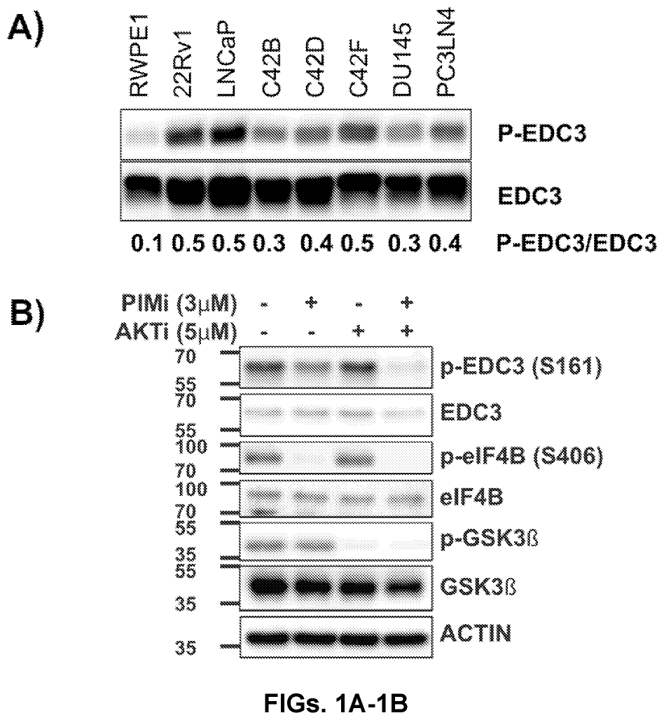

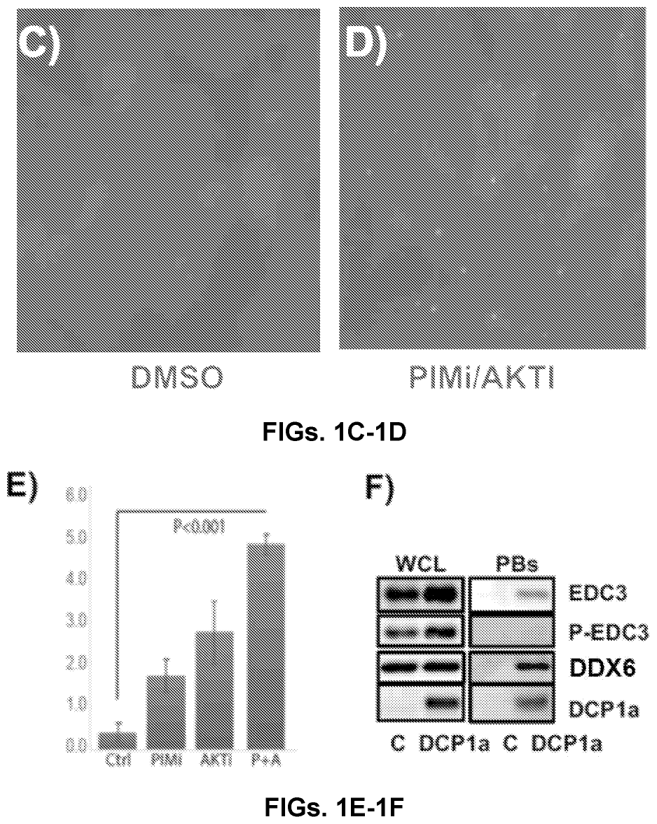

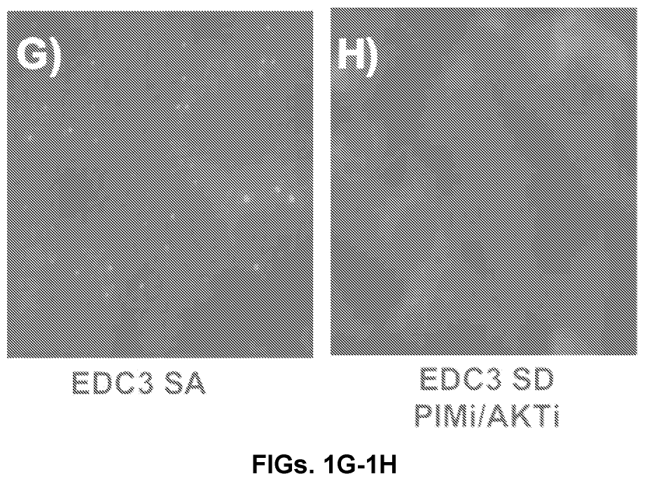

It has previously been reported that the phosphorylation of EDC3 S161 is regulated by Pim and Akt. Using an antibody specific for phosphorylated EDC3 S161 (p-EDC3), it was shown that EDC3, which contains a classic Pim kinase phosphorylation consensus RRRHNS161, is 3-5 times more highly phosphorylated in PCa cell lines (the ratio of phospho (p)/total-EDC3) compared to the non-transformed RWPE1 (FIG. 1A). A similar result was seen in breast cancer cells and leukemia, suggesting that EDC3 phosphorylation is elevated in transformed cells. In PCa cell lines PC3-LN4, the phosphorylation level of EDC3 was reduced with the Pim inhibitor Pim447 and further reduced when combined with an AKT inhibitor (AKTi) GSK690693. (FIG. 1B). In vitro kinase assays demonstrated that recombinant human Pim1 and AKT phosphorylate this site1; AKT recognizes a similar sequence RxRxxS/T to that of Pim. Thus, in prostate cancer cells, EDC3 S161 is highly phosphorylated by Pim and/or AKT when compared to non-transformed cells. Treatment of PC3-LN4 cells with a Pim and AKT inhibitor markedly increased PB number in these using PC3-LN4 expressed GFP-DCP1a (PBs are isolated by stepwise centrifugation then affinity purified using αGFP nanobody-affinity bead pulldown), it was biochemically confirmed that PBs contain DCP1a, EDC3, and DDX6, but not p-EDC3 (FIG. 1F). EDC3 S161A PC3-LN4 cells demonstrated increased numbers of EDC3 foci (FIG. 1G). This result was replicated by treatment of WT PCa cells with Pim and AKT inhibitors which induced increased PB numbers. In contrast, the EDC3 phosphomimetic S161D failed to form PBs (FIG. 1H) even when treated with inhibitors. P-bodies form in RWPE1 cells which express only tiny amounts of p-EDC3 (FIG. 1I), suggesting that dephospho-EDC3 regulates PB formation. Together these results suggest that Pim/AKT-mediated phosphorylation of EDC3 can inhibit PB formation (FIG. 1C). Therefore, it is hypothesized that dephosphorylated EDC3 plays a role in PB formation, and impacts mRNA targeting, storage, and decapping in PBs.

### Example 2: P-EDC Protein and EDC3 Levels are Higher in PCa Tumors than in Normal Controls

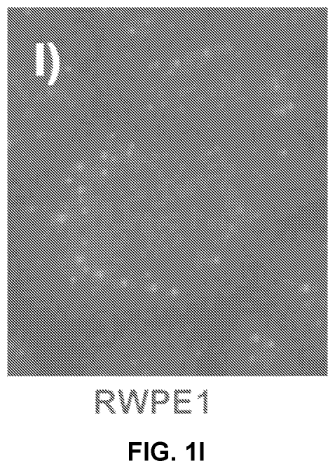

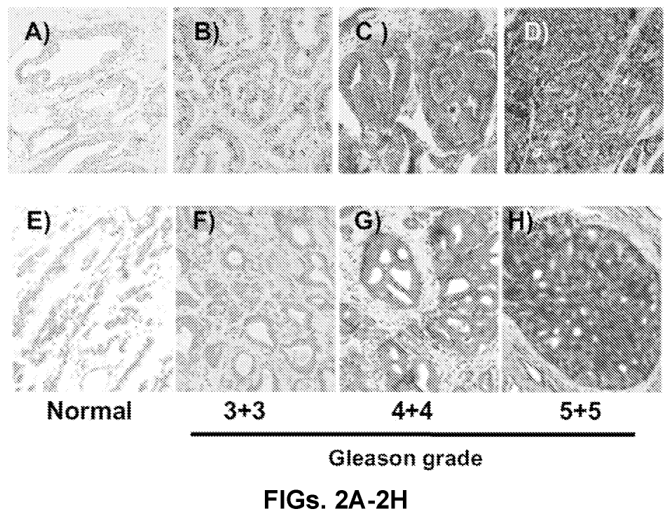

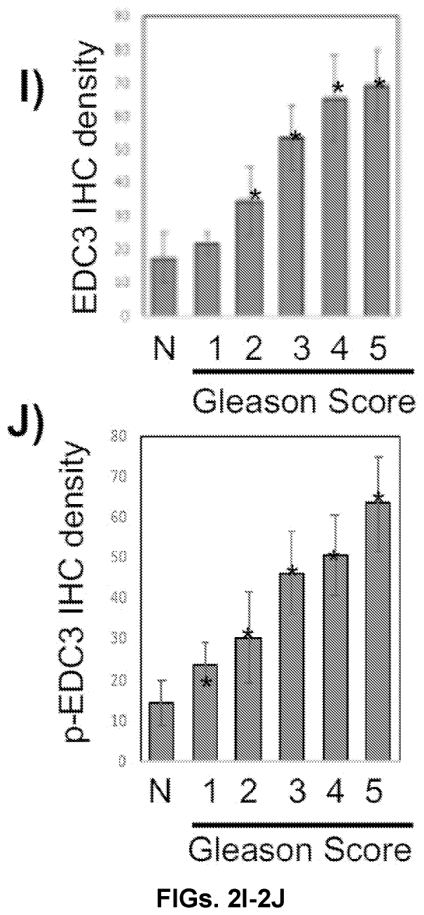

To validate the clinical importance of EDC3 and p-EDC3 in prostate IHC techniques to measure these proteins in tissue microarrays (TMAs) were established. EDC3 antibody was used for staining which was detected by 3,3′-diaminobenzidine staining (brown) and phospho-EDC3 antibody was indicated as red. The levels of EDC3 and p-EDC3 expression positively correlated with the increase of prostate cancer aggressiveness indicated by the Gleason grade (FIGS. 2A-2H). The levels of EDC3 and p-EDC3 were quantified by Dr. Sun, the group's Pathologist after examining 6 fields from 6 different patients at each Gleason grade (FIGS. 2I-2J). Thus, during PCa progression the level of both EDC3 and p-EDC3 are increased. Experiments are developed in this grant to systematically analyze whether p-EDC3 elevation identifies patients within each Gleason grade that have a poorer clinical outcome.

### Example 3: EDC3 S161A Phospho-Dead Mutation Inhibits Tumor Cell Growth, Migration, and Invasion of PCa Cell Line

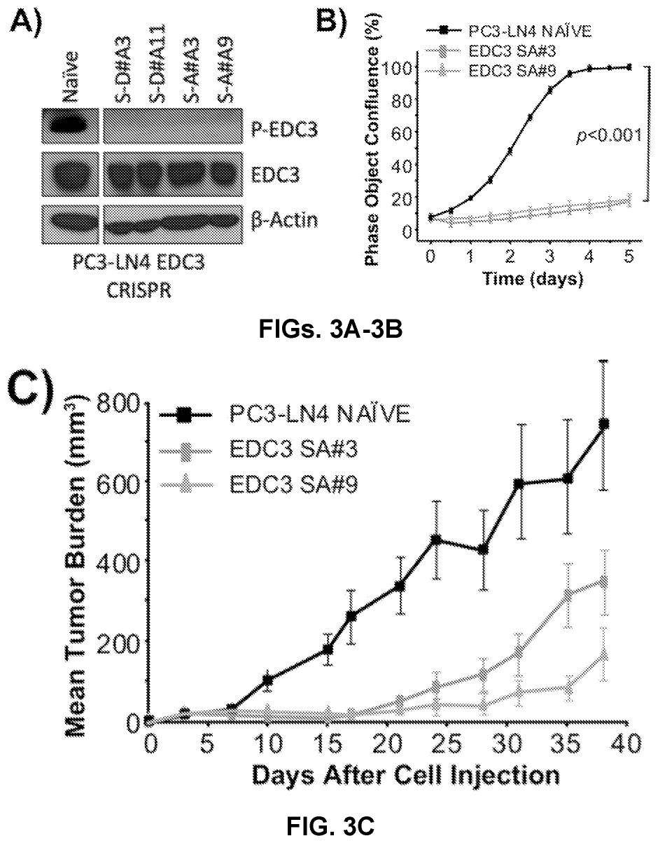

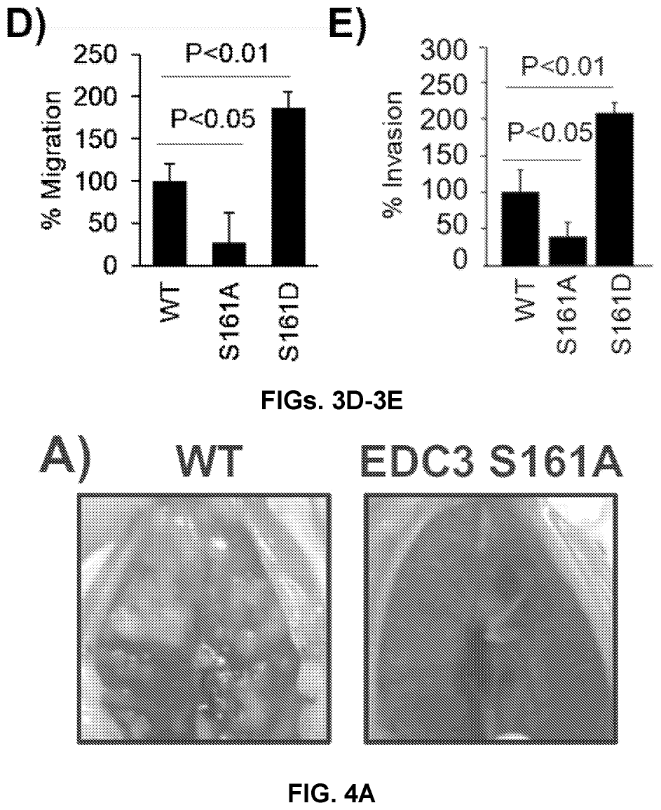

To examine the biologic importance of EDC3 S161 phosphorylation, two independent PC3-LN4 EDC3 S161A (S-A) clones were generated using the CRISPR/Cas9 system. After confirmation by sequencing, the lack of phosphorylation was validated by Western blot in each cell line (FIG. 3A). Compared to wild type (WT) both of the phospho-dead EDC3 S161A (S-A) clones grew much more slowly in culture (FIG. 3B) and when injected subcutaneously (s.c) into immunocompromised mice the mutant cells formed tumors at a much slower rate than WT (FIG. 3C). EDC3 S-A cells showed impaired ability to migrate in a tissue culture transwell migration assay, whereas phosphomimetic EDC3 S161D cells (containing a serine to aspartic acid mutation) migrated significantly better than WT cells (FIG. 3D). Similarly, transwell invasion assays revealed that EDC3 S161A cells invade significantly less than WT, while S161D mutant cells exhibit significantly more invasion than WT cells (FIG. 3E). Thus, inactivating the EDC3 phosphorylation site in PCa cells regulates significantly slower tumor growth, migration, and invasion.

### Example 4: EDC3 S161A Mutation Inhibits PCa Invasion in a Novel Mouse Diaphragm Assay

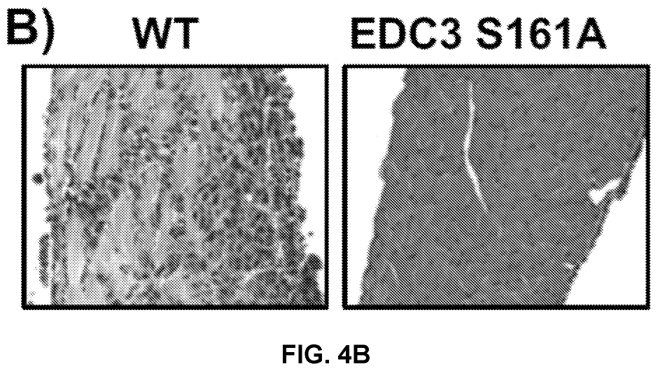

To test whether EDC3 phosphorylation is required for tumor invasion, EDC3 S161A cells were intraperitoneally injected to evaluate invasion into the smooth muscle of the diaphragm. This mimics prostate cancer invasion, since for metastasis to occur prostate tumor cells must invade the prostate capsule, which is also composed of smooth muscle. PC3-LN4 WT and EDC3 S161A were injected (5×106) into the abdomen of NSG mice and after 27 days necropsy was done. FIG. 4A shows the WT PCa cells implant onto the diaphragm but the EDC3 S161A mutant cells do not. To examine invasion microscopically, the diaphragm was fixed and then sectioned parallel to its length (FIG. 4B). H/E staining revealed that, the WT PC3-LN4 cells could attach and invade the diaphragm, while the EDC3 S161A cells could not. Thus, the phosphorylation status of EDC3 affects the ability of PCa to invade smooth muscle.

### Example 5: EDC3 S161A Cells Reduce mRNAs in Cell Adhesion, Migration, and Proliferation Pathways

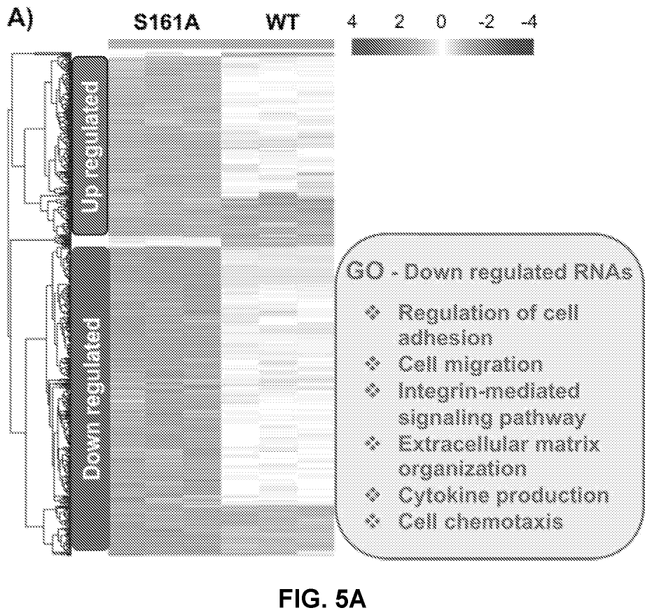

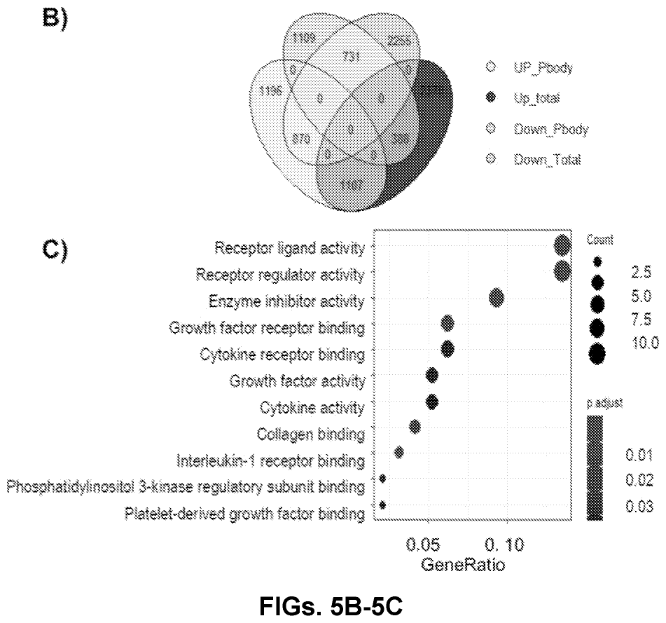

To identify genes that are controlled by EDC3, gene expression profiles of 1) total, 2) PB, and 3) uncapped mRNAs from PC3-LN4 WT and EDC3 S161A cells were analyzed, and their gene changes and signature patterns were assessed. The mRNA reads from total RNAs of PC3LN4 WT and EDC3 S-A cells indicated that EDC3 S-A mutants exhibited 6968 genes differentially expressed (FDR<0.05) when compared with WT cells. 4741 genes with the highest significance (FDR<0.01) were input for hierarchical clustering. Genes were ranked by differential expression p-value and by up- or down-regulation. Gene ontology analysis demonstrated that cell adhesion, migration, integrin signaling, and cytokine production were reduced by this phospho-inactive mutation (FIG. 5A). Analysis of PBs RNAs from EDC3 S161A showed 3372 genes overexpressed compared to WT PBs. Many of these genes are involved in mRNA catabolic processes, and RNA binding complexes, as well as translation initiation. Among the significantly differentially expressed genes (FDR corrected P value <0.05) in PBs and total RNAs from EDC3 S-A cells, 870 upregulated genes in PBs were inversely correlated to total mRNAs (FIG. 5B). The top-most significant enrichments showed changes in integrin related pathways. 3) In collaboration with Dr. Brian Gregory “Genome Wide Mapping of Uncapped and Cleaved Transcripts” (GMUCT) was performed on PC3-LN4 WT and EDC3 S161A cells. This technique sequences both uncapped and cleaved transcripts only. 117 transcripts were found that were unstable in EDC3 SA compared to WT. These transcripts were found to be enriched in receptor binding and regulator activity, signaling pathways and cytokine receptor interaction (FIG. 5C). GMUCT mRNA stability of mutant SA and WT cells was investigated using Proportion Uncapped metric. Total mapping reads were normalized to reads per million (RPM) and proportion uncapped was calculated as log 2 ratio of normalized GMUCT reads of a transcript divided by the total RNA-seq read of the corresponding transcript. Collectively, this data argues that EDC3 S161 phosphorylation has a broad effect on regulating mRNA levels that are particularly implicated in invasion and cell proliferation.

### Example 6: EDC Mutation Affects the Level of Integrin β1 (ITGB!) and α6 (ITGA6) in PCa Cells

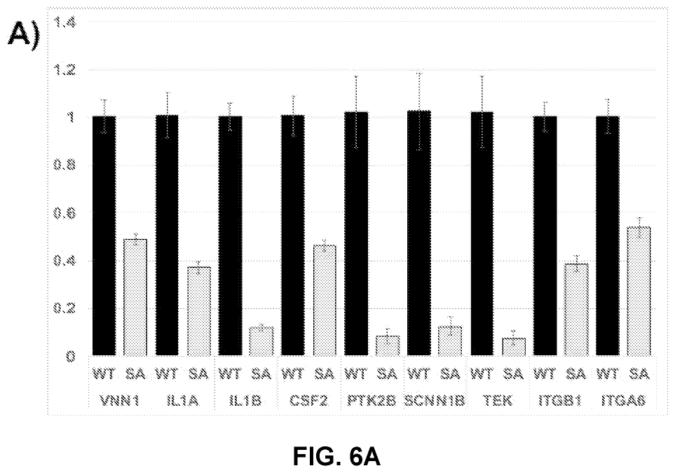

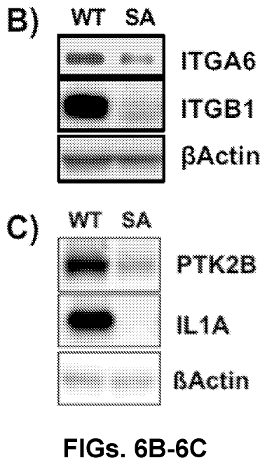

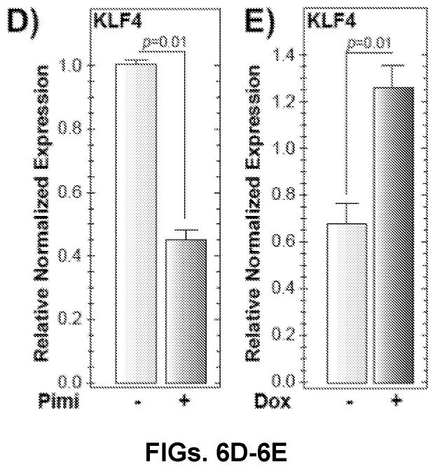

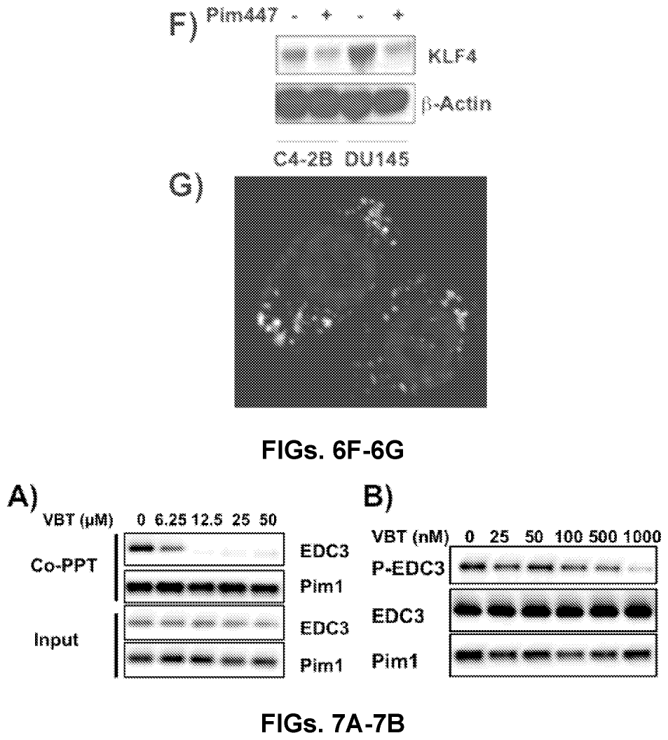

Based on genes identified in the whole cell mRNA seq analyses, it was confirmed by qPCR that the mRNAs which encode cytokines; CSF2 (GM-CSF), IL1A and IL1B, and genes involved in cell adhesion; PTK2B (Focal Adhesion Kinase 2, FAK2), TEK (TEK Receptor Tyrosine Kinase), VNN1 (Vanin 1); and Integrins (FIG. 6A) are indeed downregulated. As a reflection of this change ITGB1 and ITGA6 protein are markedly decreased in the EDC3 S-A mutant cells (FIG. 6B). Integrin α3b1 and α6b1 are needed to attach to laminin which is critical to invade smooth muscle in the prostate capsule and stimulate metastasis. These results potentially explain the lack of ability of these cells to migrate and invade both in tissue culture and animal models. The knockout of Dcp1a in these cells reverses increases in these same genes again demonstrating that they are regulated by 5′ to 3′ degradation. KLF4, a transcription factor, mRNA is observed in the purified mRNA fraction of PBs; the knock down of EDC3 elevates KLF4 mRNA levels. In this analysis, the protein and mRNA level were reduced in PC3-LN4 EDC3 S161A mutant cells. Pim447 treatment reduced KLF4 mRNA levels in PC3-LN4 cells (FIG. 6C), and protein levels in C4-2B and DU145 prostate cancer cell lines (FIG. 6E). Conversely, Pim-1 over-expression in PC3-LN4 cells increased expression of KLF4 mRNA (FIG. 6D). Using single molecule mRNA FISH (smFISH) combined with immunostaining in PC3-LN4 EDC3 S161A cells, strong colocalization between EDC3 and KLF4 mRNA is observed in PBs (FIG. 6F). EDC3 phosphorylation by Pim could regulate KLF4 levels through storage or degradation of KLF4 mRNA in PBs to impact on PCa growth. Thus, it is hypothesized that the phosphorylation of EDC3 induced by oncogenic protein kinases, Pim and AKT, plays a role in regulating tumor growth through modulating PB activity, EDC3 localization and function in mRNA decapping and translation regulation-controlling translation and/or decay of key cancer and growth relevant transcripts.

### Example 7: VBT-5445 Blocks the Binding of EDC3 to Pim1 and Inhibits the Growth of Prostate Cancer Cells

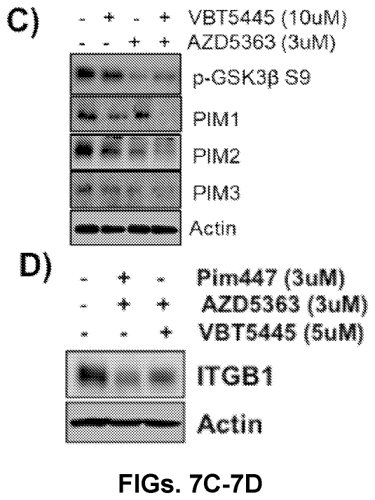

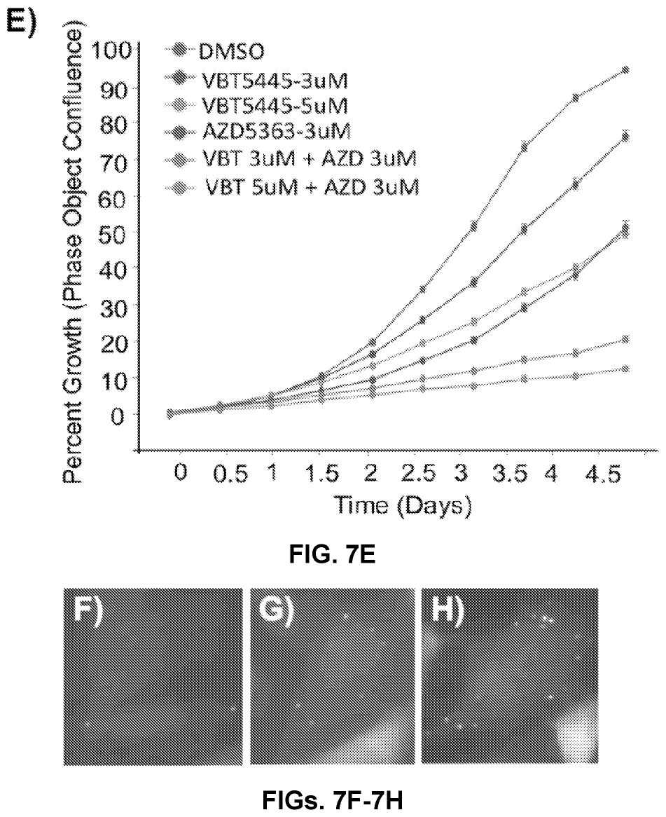

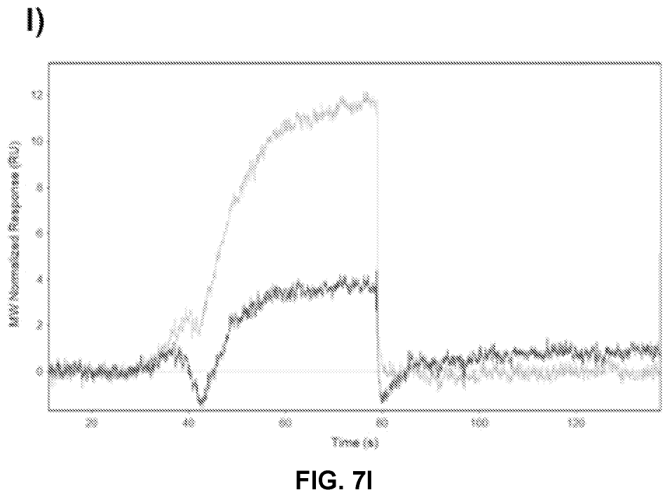

A virtual screening effort was mounted to find chemicals that interact outside of ATP binding site of Pim screening over 100,000 compounds followed by testing the top 303 by Surface Plasmon Resonance (SPR). Synthesis of the most likely candidate was achieved by the Arizona Center for Drug Discovery at the University of Arizona. In vitro, this compound was able to inhibit both the ability of EDC3 and Pim1 to coimmunoprecipitate and block the phosphorylation of EDC3 S161 by Pim (FIGS. 7A-7B). Importantly, unlike ATP competitive Pim inhibitors that increase the levels of Pim kinases after treatment (data not shown), VBT decreases the level of all Pim kinases (FIG. 7C). ITGB1 protein levels are reduced both by the combination Pim447/AZD (AKTi) and VBT/AZD (FIG. 7D). Incubation of VBT alone or in combination with the AKT inhibitor AZD5363 markedly inhibits the growth in culture of PC3-LN4 cells (FIG. 7E). VBT5445 treatment increased PB numbers, and in combination with an AKT inhibitor further increased these numbers (FIGS. 7F-7H). As shown by surface plasmon resonance (SPR) VBT5445 binding to Pim1 was increased 3-fold (FIG. 7I) when PIM447 was added and bound to the ATP site. Thus, VBT5445 competes with EDC3 for binding to Pim substrate site and is a novel inhibitor of this interaction that potentially enhances PB function by inhibiting EDC3 binding and phosphorylation.

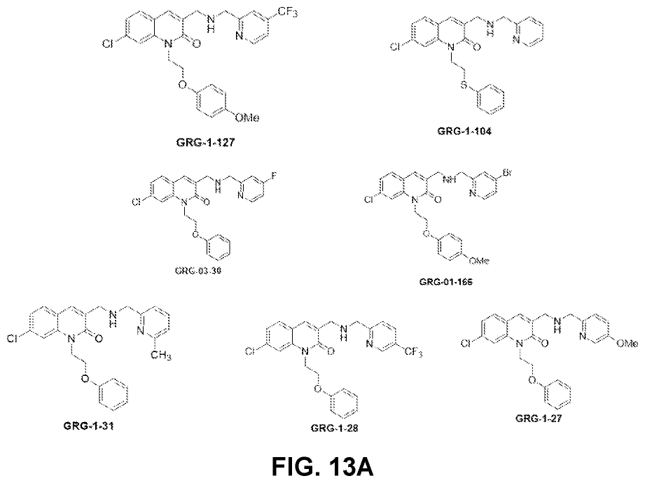

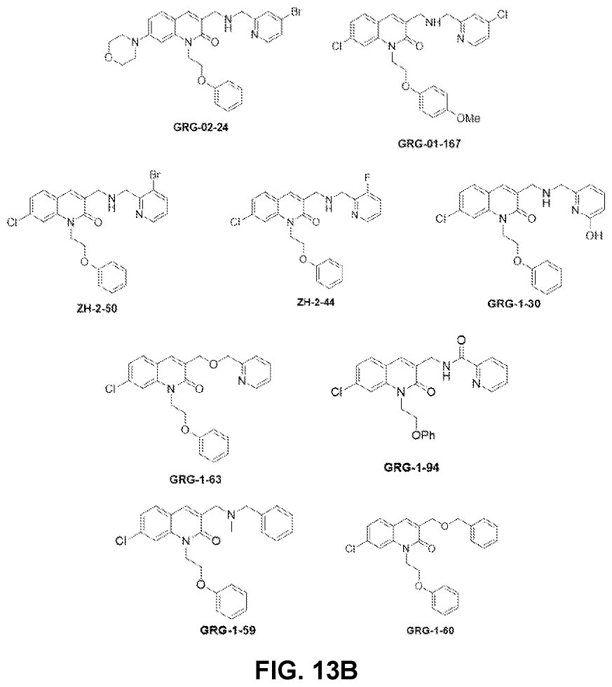

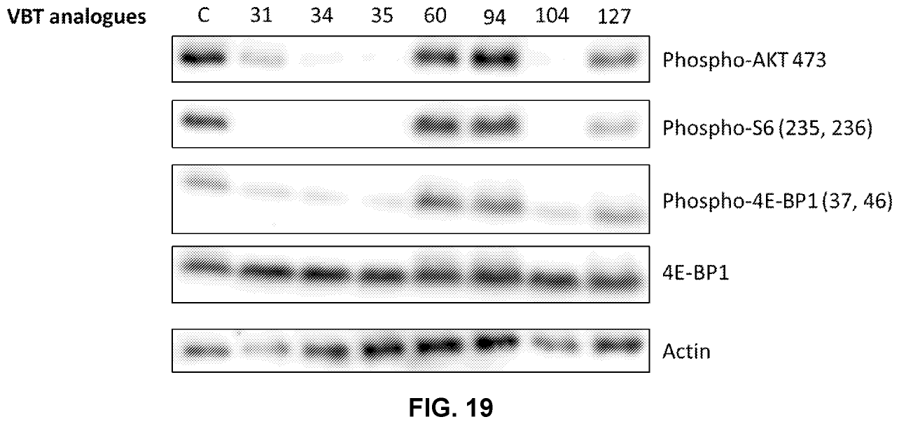

Analogs of VBT5445 were created and shown to inhibit the growth of pancreatic, prostate, leukemia, breast, and lung cancer (FIGS. 13B, 14, 15, 16, 17, and 18). These compounds also inhibited the mTOR pathway with inhibition of the phosphorylation of p70 S6kinase, 4E-BP1 and S6. In a cell line dependent manner these compounds regulated the phosphorylation of AKT on serine 473 (FIG. 19). This activation of mTOR was correlated with the inhibition of acetyl COA carboxylase and stimulation of the AMP dependent protein kinase.

### Example 8: Additional Experiments

Phosphorylated EDC3 (p-EDC3) levels as a prognostic marker of human PCa invasion and poor patient outcome. Since changes in EDC3 appear to regulate tissue invasion, using human tissue microarrays (TMAs), it will be examined whether changes in EDC3 phosphorylation and levels are a predictor of a worse clinical outcome for patients and correlate with tissue markers of invasion. TMAs obtained from the Prostate Cancer Biorepository Network (PCBN) will allow us to examine EDC3 levels and phosphorylation in patients that have both good and bad outcomes, and varying Gleason grades and stage. Techniques allowing the detection by immunohistochemistry (IHC) of total-EDC3 and phospho (p)-EDCS161 on tissue microarrays (TMA) of human PCa are described in preliminary results. To evaluate the results, these slides will be scored on a 4+, 3+, 2+, 1+, 0 basis. Preliminary results have identified proteins whose level drops when EDC3 is dephosphorylated. Using IHC, it will be determined whether these proteins correlate with p-EDC3 and have poor outcomes.

To assess whether p-EDC3 levels and its targets are correlated with poor prognosis and death in PCa, the p-EDC3 status will be assessed in a TMA that includes patient outcomes. The first TMA to be studied is constructed from 235 cases previously described in the literature. These prostatectomies were done over a 15-year period and include patients who recurred and developed metastatic or did not have recurrent disease. The initial Gleason grade and PSA are identified for each prostatectomy specimen. These slides will be stained for both EDC3 and p-EDC3 and examined for a correlation between poor outcome and changes in EDC3. Next, using IHC the level of genes known to be regulated by p-EDC3 in culture, ITGB1, ITGA6, PTK2B (FAK2), and IL1a will be measured and correlated with p-EDC3 and EDC3 levels. If for technical reasons these proteins cannot be evaluated, others potential targets, e.g., CSF2 (GM-CSF) or TEK, will be studied.

To validate and expand these observations, a significantly larger PCBN TMA from Johns Hopkins that contains 524 cases will be used. In this TMA, for each prostatectomy that has recurred, a control (n=524) specimen is provided that contains cancer, but the patient has not experienced recurrence. The two specimens are matched by age, race, pathological stage (T2, T3, T3b, or worse), and Gleason sum (<6, 6, 7, 8+). These TMAs will be stained for p-EDC3, EDC3, and the proteins identified above. This TMA will validate the results of the smaller one but also help us specifically examine whether at any Gleason score those patients with higher level of EDC3 or p-EDC3 have a worse outcome than the other patients with an identical score. This analysis will tell us whether for each Gleason grade high p-EDC3 identifies patients with a worse prognosis and is correlated with elevations in proteins associated with invasion and cell signaling.

How increases in p-EDC3 regulate RNA degradation. By understanding the mechanism by which alteration of p-EDC3 and EDC3 levels affects RNA degradation, it will be possible to target these pathways to develop new approaches to cancer treatment.

How changes in EDC3 levels and in phosphorylation control RNA metabolism and thus tumor invasion is unknown. To elucidate the mechanism by which phosphorylation effects mRNA decapping, PB formation, and RNA decay, this aim will explore three novel possibilities: 1) whether phosphorylation alters the affinity of the EDC3 protein complexes for specific mRNAs preventing RNAs from entering the decapping pathway, 2) whether phosphorylation of EDC3 prevents self-dimerization and binding to the decapping complex, and 3) whether EDC3 phosphorylation controls the rate of RNA decapping and by this mechanism slows the rate of degradation.

For these studies human PCa cell lines PC3-LN4 cells and C4-2B cells will be used because both have activated AKT, increased Pim kinase, and highly phosphorylated EDC3 as well as high stoichiometric phosphorylation of EDC3 observed by phospho-tag analysis. If results with these two are inconsistent, additional cell lines have been identified with increased p-EDC3 will be examined (FIGS. 1A-11).

Does phosphorylation of EDC3 in prostate tumors block specific mRNAs from being degraded via the 5′ to 3′ pathway? It is possible that p-EDC3 physically binds or holds on to a specific set of mRNAs in the cytosol preventing them from associating with PBs where they would be degraded. Dephosphorylating EDC3 could free these mRNAs to associate with PBs and thus be either be decapped or stored. To determine if dephosphorylation of EDC3 allows RNAs to move from the cytosol to the PB ITGB1, PTK2B (FAK2) and IL1a will be examined (FIGS. 6A-6G), all of which decrease markedly when EDC3 is dephosphorylated. Single molecule (sm) RNA FISH will be combined with immunohistochemistry for EDC3 and Dcp1a, part of the decapping complex, to identify P-bodies. smFISH probes that contain 48 different regions of these genes have been designed in conjunction with Biosearch Technologies and labeled with Quasar 670 dye. P-bodies will be stained with anti-Dcp1a and EDC3 antibody. To examine whether dephosphorylation of EDC3 sends these mRNAs from the cytosol to be the PBs, tumor cells will be treated with AZD5363 (3 μM) or Pim447 (3 μM) or the combination for 6 h and smFISH performed. If this theory is correct, then analysis of PC3LN4 with knock-in of EDC3 S-A or EDC3 S-D will find these mRNAs in opposite locations.

To test if the mRNAs initially identified from the bioinformatics analysis physically bind to EDC3 and its protein complex RNA and protein will be cross-linked. Using CLIP techniques, EDC3 will be immunoprecipitated, and mRNAs extracted from the immunoprecipitant. These mRNAs will be analyzed by qRT-PCR focusing initially on the mRNAs that have been shown are decreased in the EDC3 S-A cells, CSF2 (GM-CSF), IL1a, IL1b, PTK2B (FAK2), TEK, VNN1, ITGB1 and ITGA6. Additionally, to confirm that this protein or the decapping complex directly interacts with a specific mRNA in vivo, RNA Antisense Purification (RAP) analysis will be used, which purifies a complex containing mRNA and the interacting proteins. Briefly, as with CLIP, after UV crosslinking and fractionation, mRNA/protein complexes are purified with biotin-labeled oligo DNA probes to the mRNAs using denaturing conditions to disrupt non-covalent interactions. The detection of proteins in the RAP analysis will be done by mass spectrometry.

To explore the nature of the bound RNAs in greater detail, high throughput sequencing of RNAs isolated by Photoactivatable-Ribonucleoside-Enhanced Crosslinking and Immunoprecipitation (PAR-CLIP) will be carried out. This method utilizes nontoxic levels of the uracil analog 4-thiouridine combined with 365 nm irradiation to efficiently form cross links

between labelled RNAs and aromatic amino acid side chains of bound proteins. In this technique uracil analog labelling is pulsed and followed by iodoacetamide treatment and reverse transcription. A time course will be done after the pulsing. The PAR-CLIP data will be analyzed with the wavClusteR (R package v2.22.0) pipeline and compare bound mRNAs in the phosphorylated EDC3 complex with those treated with kinase inhibitors as described previously. Again, PCa cells containing either a EDC3 S-A or S-D mutation will be analyzed and compared to WT and treated cells. Bioinformatic analysis will use sequence analysis, motif binding analysis (MEME) RNA binding proteins databases and gene ontology terms enrichment to find the classes of mRNA bound to this complex. It will be determined whether these mRNAs contain a specific length or consensus sequence or RNA structure in their 5′ or 3′ untranslated region or whether they contain specific 3′ sequences that are recognized by 3′ binding proteins especially those known to be associated with PBs (e.g., TTP and PUM1). These analyses will define the classes of genes that are regulated by phosphorylation and dephosphorylation of EDC3.

Do increased levels of p-EDC3 and EDC3 in tumor cells sequester proteins that regulate RNA decapping and degradation? EDC3 is known to scaffold proteins that bind 5′ decapping including the decapping enzymes Dcp2 and Dcp1a and the translation regulator DDX6. Phosphorylation of EDC3 could a) make this protein unable to bind to other decapping proteins or dimerize blocking its ability to enhance RNA degradation or b) alternatively, EDC3 could act as a “sponge” and soak up all proteins needed for 5′ decapping but be unable to associate with PBs. To examine these two opposite possibilities, using PC3-LN4 and/or C4-2B that overexpress GFPDcp1a, this fusion will be immunoprecipitated (IP) and the amount of p-EDC3 and EDC3 in the co-IP samples will be assessed by Western blotting using Abs that recognize phospho- or pan EDC3. Alternatively, GFP-EDC3 will be immunoprecipitated from PC3-LN4 and/or C4-2B prostate cancer cells and the amount of Dcp1a, Dcp2 and DDX6 bound evaluated by Western blotting. To assess whether binding changes with EDC3 dephosphorylation treatment, PCa cells will be treated with Pim-447 (3 μM) and AZD5363 (3 μM) or DMSO for 6 h and the change in the proteins bound to GFP-Dcp1a or GFP-EDC3 assessed. Additionally, using PC3-LN4 and C4-2B cells that express GFP-EDC3 S-D protein it will be examined whether the EDC3 S-D binds decapping factors preventing PB formation pointing to the sponge effect. If pEDC3 does not bind these proteins and is not a “sponge”, but dephosphorylation actually induces binding especially in PBs, then phosphorylation could cause structural changes in the molecule.

The possibility that phosphorylation induces structural changes in EDC3 that could affect its protein interactions will also be tested. NMR spectroscopy will be carried out following Pim1 phosphorylation. The team will record 2D [¹H,¹⁵N]HSQC spectra to follow changes in the EDC3 spectrum. Ser/Thr phosphorylation can be readily followed as it induces large, characteristic chemical shift changes in the phosphorylated residues. Small Angle X-ray scattering (SAXS) can be used as an alternative method to probe effect of phosphorylation on EDC3 structure, as described for Dcp1/Dcp243. These experiments will demonstrate if phosphorylation of EDC3 induces significant changes in its conformation, so that it can no longer bind Dcp1 or effects its ability to form dimers, both of which may influence its ability to promote decapping. The former possibility will be tested by direct binding assays using short linear motifs from Dcp1 known to bind the Lsm domain of Edc3 using NMR or fluorescence anisotropy as described in the literature. The latter possibility will be tested using size-exclusion chromatography or analytical ultra-centrifugation.

Does EDC3 S161 phosphorylation block the ability of EDC3 to enhance the rate of decapping? It remains possible that EDC3 phosphorylation may not alter EDC3's affinity for decapping factors or specific mRNAs, but instead inhibit the decapping activity of the complex. It has been shown that yeast EDC3 induces a 90× increase in RNA decapping in condensates. The effect of phosphorylation on condensate formation will be evaluated. The concentration of Dcp1 and Dcp2 needed for droplet formation will be examined in the absence of EDC3, or in the presence of WT EDC3, S-A, and S-D, a phosphomimic. Pim1 will be used to phosphorylate EDC3 in the presence of ATP and Mg²⁺ and it will be evaluated whether this phosphorylation or the presence of Pim1, which binds EDC3 tightly, interferes with the incorporation of Edc3 into condensates.

To measure rates of decapping in condensates, an RNA substrate containing a 5′-cap conjugated to fluorescein and 3′ adenosine conjugated to Cy5 will be used. Loss of M7G fluorescein is dependent on catalytic activity of Dcp2 regulated by EDC3. Using this reagent, the effect of phosphorylation on the rate constant for decapping in these condensates described above will be evaluated. The above experiments will allow us to quantify the degree of activation of decapping by Edc3 in condensates and in dilute phase (below the critical concentration for liquid-liquid phase separation).

Another in vitro approach to examining whether phosphorylation blocks the ability of EDC3 to enhance decapping of mRNAs is to make use of classical in vitro decapping assays. These assays will be performed

using purified GST-EDC3, both wild type protein and EDC S161A and S161D, mRNA decapping enzymes (Dcp1a, Dcp2), and cap-radiolabeled ITGB1 or PTK2B substrates. Assays will be performed with the Pim kinase plus ATP/Mg++ or ATP/Mg++ alone to assess the impact of phosphorylation on decapping activity.

To analyze this question in PCa cells, PC3-LN4 and C4-2B cells containing WT EDC3, EDC3-S-A, and S-D (CRISPR knock-in) will be treated with actinomycin D to obtain transcription shutoff and then at specific times after treatment (0.5, 1, 2, 4, 6 h), RNA will be isolated and analyzed by splinted ligation reverse transcription polymerase chain reaction, qSL-RT-PCR50. The rate of decapping of CSF2 (GM-CSF), IL1a, IL1b, PTK2B, TEK, VNN1 and ITGB1 and ITGA6 will be investigated. Total mRNA levels will also be assessed with qRT-PCR and estimate the fold changes under each condition. Importantly, in WT cells the rate of decapping of these RNAs will be examined with and without Pim and AKT inhibitor treatment to delineate whether dephosphorylation of EDC3 changes decapping rates.

Use of small molecule drugs to regulate RNA destruction in tumors in vivo. These experiments will determine whether a novel small molecule Pim1 substrate competitor, with or without an AKT inhibitor, can regulate mRNA decay in PCa tumors and whether this stimulation of mRNA decapping results in inhibition of growth and invasion. Developing a small molecule that interferes with the substrate binding pocket of Pim and prevents EDC3 binding is a novel approach to PCa treatment. This approach has the potential to yield increased target specificity and fewer off target effects when compared to kinase inhibitory compounds that bind in the ATP pocket. In the clinic it is difficult to administer two kinase inhibitors together because of the combined side effects. Preliminary results suggest that VBT treatment of PCa induces RNA decapping and degradation, increases PB numbers, and when administered with the AKT inhibitor AZD5363 significantly blocks PCa growth while decreasing the level of ITGB1. In human phase II clinical trials, AKT and PI3K inhibitors are being administered to PCa patients, but have not had impressive clinical success, suggesting that there is a complete understanding of how these agents work may be lacking. To impact on patient outcomes, two important and relevant questions will be addressed: 1) In human PCa PDX models can kinase inhibitors when administered alone, e.g., AKT inhibitor, or in combination induce the decapping and decay of a specific set mRNAs that control invasion, and 2) Are these kinase inhibitors capable of blocking tumor invasion? These experiments will determine whether drugs administered to tumor bearing mice will replicate the anti-tumor activity of the CRISPR knock-in of EDC3 S-A.

Dose and frequency of administration for Small Molecule Pim Kinase Inhibitors. To determine the best frequency of dosing for VBT, the half-life of this compound in mice will be measured when administered with or without the AKT inhibitor AZD5363. Balb/c mice (3/group) will be gavaged singly with VBT5445 (50 mg/kg) in Cremophore/Ethanol/Water—24/6/70 ratio or in combination with AZD5363 (40 mg/kg) at times 0, 0.25, 0.5, 1, 2, 4, 8 and 12 h, the animals will be sacrificed, and the blood harvested. The plasma will be extracted, and the drug levels analyzed by HPLC with MS detection. Pharmacokinetic parameters in the blood will be calculated. By learning the half-life and AUC, it will be possible to determine how frequently this agent should be administered for maximal effect.

Maximum Tolerated Dose and Side Effect Profile. It has been shown that VBT5445 can be given safely at a dose of 30 mg/kg by gavage in combination with AZD5363 40 mg/kg without any weight loss or behavior changes in mice. To obtain the maximum tolerated dose (MTD), the dose will be increased by 50% over the previous one at each ensuing treatment consisting of 3 mice given daily for 5 days. Mice will be observed for fur ruffling and behavior changes. Blood will be drawn, and the WBC count and blood chemistry will be determined to evaluate bone marrow, liver, and kidney toxicity. Together with pharmacokinetic profiling, this analysis will help determine the best dose and frequency of administration for this molecule.

Treatment of in vivo tumors with small molecule Pim kinase inhibitors, an AKT inhibitor, or both and the decapping of tumor RNA. These experiments will be carried out using human PCa PDX tumors (named as a group LuCAP). These tumors mirror the variability in AKT and Pim levels seen in human cancers. To assess whether treatment with these agents regulates the decapping of a specific biologically functional group of mRNAs identified in preliminary results, LuCaP 93, 136, containing high levels of Pim and activated AKT secondary to PTEN deletion and Pim overexpression and LuCaP 78 with normal levels of both kinases will be grown subcutaneously in NSG mice. Using mice with varied levels of Pim and AKT will allow us to evaluate the importance of measuring these levels when examining treatment outcomes. Mice harboring tumors will be treated in groups of 5 and gavaged once daily with VBT5445 (MTD), AZD5363 (40 mg/kg) or the combination. After 1 week of treatment tumors will be removed, sent for pEDC3 IHC to document dephosphorylation and mRNA extracted and submitted for RNA seq. and GMUCT, “Genome Wide Mapping of Uncapped and Cleaved Transcripts”. This analysis will allow us to determine whether these treatment(s) are modulating RNA decapping. mRNA stability of mutant S-A and WT cells will be investigated using Proportion Uncapped metric. Total mapping read will be normalized to reads per million (RPM) and proportion uncapped was calculated as log 2 ratio of normalized GMUCT reads of a transcript divided by the total RNA-seq read of corresponding transcript31. The RNA seq read counts per gene will be determined using feature counts in Rsubread pair-wise differential expression will be performed viaDESeq256. To evaluate the changes induced whole cell RNA seq the highly significant (FDR<0.01) genes will be taken as input for hierarchical clustering. Statistically significant clusters of RNAs will evaluated for functional and pathway enrichment.

Bioinformatics analysis will allow us to discover: 1) whether these inhibitors affect EDC3 dependent pathways to markedly change decapping and decay 2) whether these decapped RNAs are identical or overlapping with potential EDC3 targets e.g., GM-CSF, IL1a, IL1b, PTK2B, TEK, VNN1 and ITGB1 and ITGA6, from preliminary results 3) if the alteration of mRNA expression reflects regulatory gene clusters in cell attachment, invasion, and growth and motility and 4) if the drug combination functions better than each agent alone.

Small molecules with or without AZD5363 and PCa invasion into smooth muscle. To test whether VBT or AZD alone or in combination inhibits invasion, the three LuCAP tumors will be dissociated into single cells. To create a single cell suspension PDX tumors will be minced in DMEM/F12 media and digested with collagenase type 1 plus DNAse, and the dissociated cells will be collected by using an Amaxa Dissociator as carried out by the Corey group (letter). 5×10⁶ cells will be intraperitoneally injected into male NSG mouse. Groups of 5 NSG mice will be gavaged with VBT-5445 (MTD), AZD 5363 (40 mg/kg) or the combination. After 21 days of treatment or the time needed for mice to lose 10% body weight, mice will be necropsied, and invasion of the diaphragm observed visually. After embedding the diaphragm in a paraffin block, strips will be cut oriented perpendicular to the block and stained with H/E. The number of invasive sites and invasive depth will be measured and quantitated. IHC staining with EDC3 total and pEDC3 Abs will be carried out. Western blots will be carried out to identify a decrease in Pim kinase levels. If VBT5445 is highly specific to EDC3 then other Pim substrates should not be dephosphorylated. IRS1, elF4B, and DEPDC5 have been identified as Pim substrates highly phosphorylated in PCa. Western blots will be carried out to evaluate the level of phosphorylation of these proteins.

### Example 9: General Synthetic Methods

General Procedure for Reductive Amination: To an oven-dried RB flask containing 2-Oxoquinoline-3-carbaldehyde (1 eq) in dichloroethane was added respective amine (1-1.1 eq) followed by the addition of Sodium triacetoxyborohydride (2.5 eq) and a catalytic amount of acetic acid. The reaction mixture was stirred overnight at room temperature under a nitrogen atmosphere. After the complete consumption of starting material, the reaction mixture was quenched by the addition of water and extracted with dichloromethane. The organic phase was washed with brine and dried over Na₂SO₄, and concentrated. Further purification was carried out by either flash chromatography or preparatory-scale HPLC to isolate the target compounds (GRG/VBT compounds) in 10-62% yield as sticky yellow solids. The general procedure is summarized in Scheme 1:

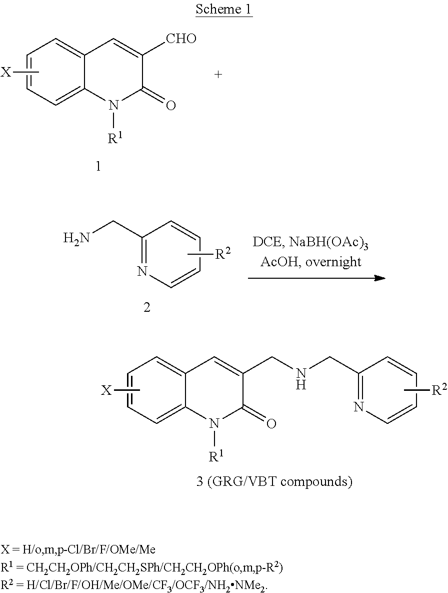

General Procedure for Amide Derivatives: (A) To an oven-dried round bottom flask containing 3-(aminomethyl) quinolin-2 (1H)-one derivative (1 eq) in dichloromethane was added respective carboxylic acid (1-1.1 eq) followed by the addition of EDC·HCl (1.5-2 eq). The reaction mixture was stirred at room temperature under a nitrogen atmosphere for 4-6 hours. After the complete consumption of starting material water was added and extracted with dichloromethane. The organic phase was washed with brine and dried over Na₂SO₄, and concentrated. Further purification was carried out by either flash chromatography or preparatory-scale HPLC to get the target compounds series 6 (GRG/VBT compounds) as white solids.

(B) Compounds of series 8 (GRG/VBT compounds) can be obtained by coupling derivatives of 2-oxo-1,2-dihydroquinoline-3-carboxylic acid (1 eq) with a respective amine (1-1.5 eq) dissolved in DCM in presence of EDC·HCl (1.5-2 eq). Workup and purification as steps as in (A). The general procedure is summarized in Scheme 2:

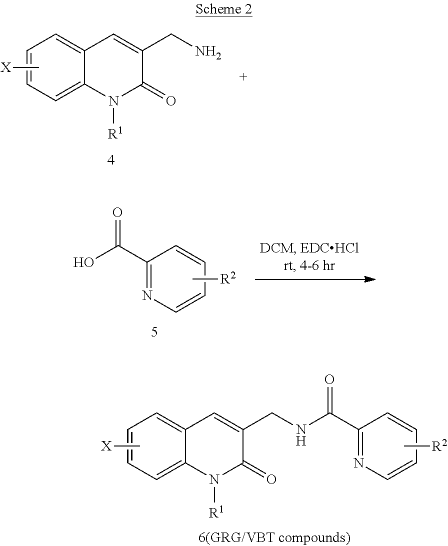

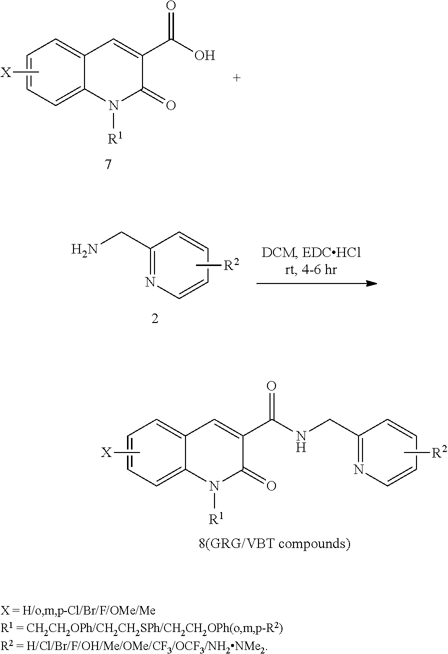

### Example 10: Small Molecule Kinase Inhibitors

Additional small molecule Pim kinase inhibitors are provided in Table 1:

TABLE 1
Small Molecule Pim Kinase Inhibitors

| S.N. | Structure | Index | Comment |
|---|---|---|---|
| | 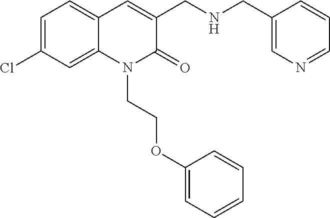 [SMILES: O=c1c(CNCc2cccnc2)cc2ccc(Cl)cc2n1CCOc1ccccc1] | VBT5445 | |
| 1 | 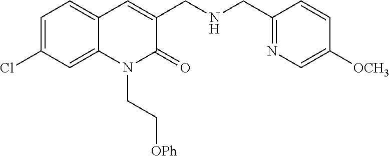 [SMILES: COc1ccc(CNCc2cc3ccc(Cl)cc3n(CCOc3ccccc3)c2=O)nc1] | GRG-1-27 (also referred to as VBT-1-27 and compound 27) | Chemical Formula: C₂₅H₂₄ClN₃O₃ Exact Mass: 449.151 |
| 2 | 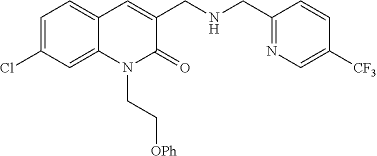 [SMILES: O=c1c(CNCc2ccc(C(F)(F)F)cn2)cc2ccc(Cl)cc2n1CCOc1ccccc1] | GRG-1-28 (also referred to as VBT-1-28 and compound 28) | C₂₅H₂₁ClF₃N₃O₂ Exact Mass: 487.127 |
| 3 | 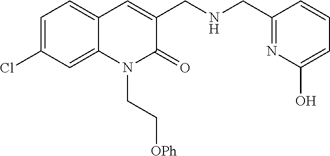 [SMILES: O=c1c(CNCc2cccc(O)n2)cc2ccc(Cl)cc2n1CCOc1ccccc1] | GRG-1-30 (also referred to as VBT-1-30 and compound 30) | Chemical Formula: C₂₄H₂₂ClN₃O₃ Exact Mass: 435.135 |
| 4 | 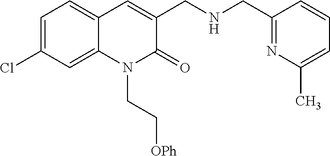 [SMILES: Cc1cccc(CNCc2cc3ccc(Cl)cc3n(CCOc3ccccc3)c2=O)n1] | GRG-1-31 (also referred to as VBT-1-31 and compound 31) | Chemical Formula: C₂₅H₂₄ClN₃O₂ Exact Mass: 433.156 |
| 5 | 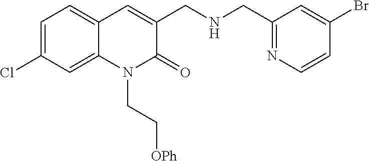 [SMILES: O=c1c(CNCc2cc(Br)ccn2)cc2ccc(Cl)cc2n1CCOc1ccccc1] | GRG-1-34 (also referred to as VBT-1-34 and compound 34) | Chemical Formula: C₂₄H₂₁BrClN₃O₂ Exact Mass: 497.051 |
| 6 | 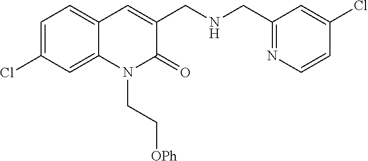 [SMILES: O=c1c(CNCc2cc(Cl)ccn2)cc2ccc(Cl)cc2n1CCOc1ccccc1] | GRG-1-35 (also referred to as VBT-1-35 and compound 35) | Chemical Formula: C₂₄H₂₁Cl₂N₃O₂ Exact Mass: 453.101 |
| 7 | 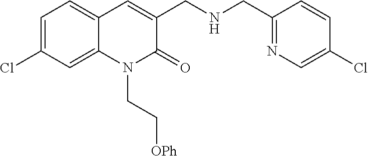 [SMILES: O=c1c(CNCc2ccc(Cl)cn2)cc2ccc(Cl)cc2n1CCOc1ccccc1] | GRG-1-39 (also referred to as VBT-1-39 and compound 39) | Chemical Formula: C₂₄H₂₁Cl₂N₃O₂ Exact Mass: 453.101 |
| 8 | 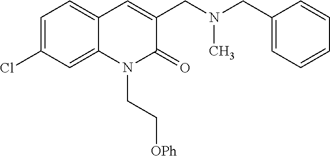 [SMILES: CN(Cc1ccccc1)Cc1cc2ccc(Cl)cc2n(CCOc2ccccc2)c1=O] | GRG-1-59 (also referred to as VBT-1-59 and compound 59) | Chemical Formula: C₂₆H₂₅ClN₂O₂ Exact Mass: 432.160 |
| 9 | 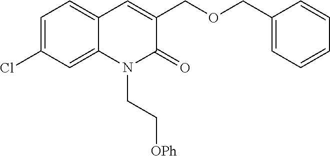 [SMILES: O=c1c(COCc2ccccc2)cc2ccc(Cl)cc2n1CCOc1ccccc1] | GRG-1-60 (also referred to as VBT-1-60 and compound 60) | Chemical Formula: C₂₅H₂₂ClNO₃ Exact Mass: 419.129 |
| 10 | 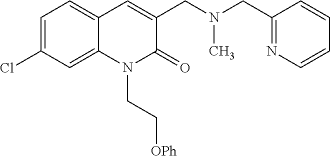 [SMILES: CN(Cc1ccccn1)Cc1cc2ccc(Cl)cc2n(CCOc2ccccc2)c1=O] | GRG-1-61 (also referred to as VBT-1-61 and compound 61) | Chemical Formula: C₂₅H₂₄ClN₃O₂ Exact Mass: 433.156 |
| 11 | 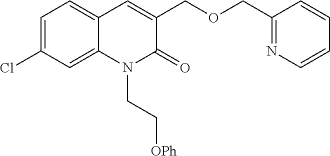 [SMILES: O=c1c(COCc2ccccn2)cc2ccc(Cl)cc2n1CCOc1ccccc1] | GRG-1-63 (also referred to as VBT-1-63 and compound 63) | Chemical Formula: C₂₄H₂₁ClN₂O₃ Exact Mass: 420.124 |
| 12 | 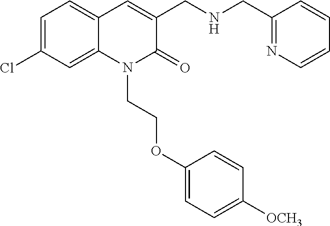 [SMILES: COc1ccc(OCCn2c(=O)c(CNCc3ccccn3)cc3ccc(Cl)cc32)cc1] | GRG-1-70 (also referred to as VBT-1-70 and compound 70) | Chemical Formula: C₂₅H₂₄ClN₃O₃ Exact Mass: 449.151 |
| 13 | 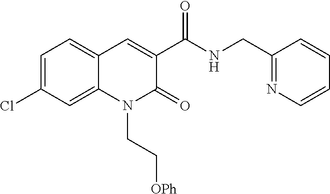 [SMILES: O=C(NCc1ccccn1)c1cc2ccc(Cl)cc2n(CCOc2ccccc2)c1=O] | GRG-1-75 (also referred to as VBT-1-75 and compound 75) | Chemical Formula: C₂₄H₂₀ClN₃O₃ Exact Mass: 433.119 |
| 14 | 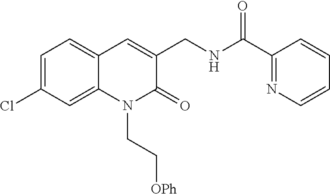 [SMILES: O=C(NCc1cc2ccc(Cl)cc2n(CCOc2ccccc2)c1=O)c1ccccn1] | GRG-1-94 (also referred to as VBT-1-94 and compound 94) | Chemical Formula: C₂₄H₂₀ClN₃O₃ Exact Mass: 433.119 |
| 15 | 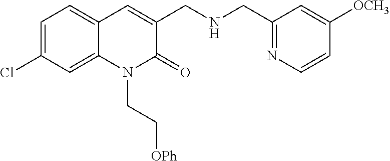 [SMILES: COc1ccnc(CNCc2cc3ccc(Cl)cc3n(CCOc3ccccc3)c2=O)c1] | GRG-1-95 (also referred to as VBT-1-95 and compound 95) | Chemical Formula: C₂₅H₂₄ClN₃O₃ Exact Mass: 449.15 |
| 16 | 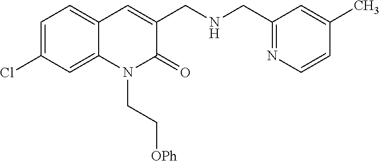 [SMILES: Cc1ccnc(CNCc2cc3ccc(Cl)cc3n(CCOc3ccccc3)c2=O)c1] | GRG-1-96 (also referred to as VBT-1-96 and compound 96) | Chemical Formula: C₂₅H₂₄ClN₃O₂ Exact Mass: 433.156 |
| 17 | 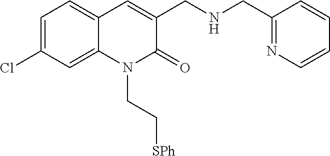 | GRG-1-104 (also referred to as VBT-1-104 and compound 104) | Chemical Formula: C₂₄H₂₂ClN₃OS Exact Mass: 435.117 |
| 18 | 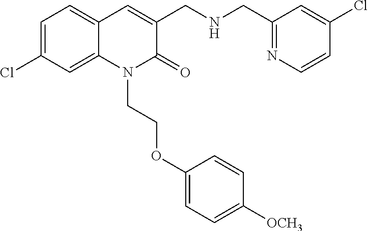 [SMILES: COc1ccc(OCCn2c(=O)c(CNCc3cc(Cl)ccn3)cc3ccc(Cl)cc32)cc1] | GRG-1-167 (also referred to as VBT-1-167 and compound 167) | Chemical Formula: C₂₅H₂₃Cl₂N₃O₃ Exact Mass: 483.112 |
| 19 | 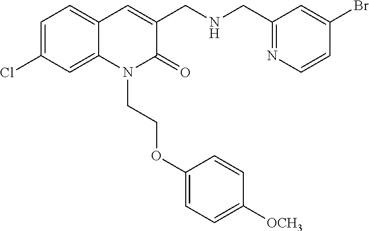 [SMILES: COc1ccc(OCCn2c(=O)c(CNCc3cc(Br)ccn3)cc3ccc(Cl)cc32)cc1] | GRG-1-166 (also referred to as VBT-1-166 and compound 166) | Chemical Formula: C₂₅H₂₃BrClN₃O₃ Exact Mass: 527.061 |
| 20 | 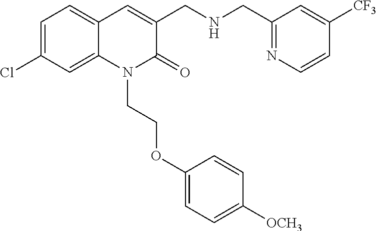 [SMILES: COc1ccc(OCCn2c(=O)c(CNCc3cc(C(F)(F)F)ccn3)cc3ccc(Cl)cc32)cc1] | GRG-1-127 (also referred to as VBT-1-127 and compound 127) | Chemical Formula: C₂₆H₂₃ClF₃N₃O₃ Exact Mass: 517.138 |
| 21 | 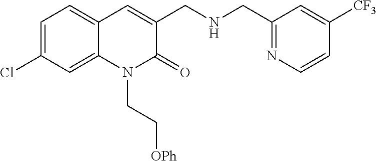 [SMILES: O=c1c(CNCc2cc(C(F)(F)F)ccn2)cc2ccc(Cl)cc2n1CCOc1ccccc1] | GRG-1-128 (also referred to as VBT-1-128 and compound 128) | Chemical Formula: C₂₅H₂₁ClF₃N₃O₂ Exact Mass: 487.127 |
| 22 | 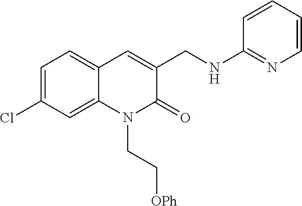 [SMILES: O=c1c(CNc2ccccn2)cc2ccc(Cl)cc2n1CCOc1ccccc1] | GRG-1-153 (also referred to as VBT-1-153 and compound 153) | Chemical Formula: C₂₃H₂₀ClN₃O₂ Exact Mass: 405.124 |
| 23 | 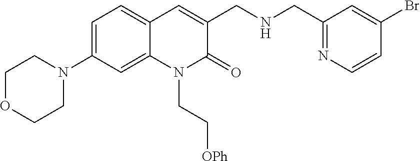 [SMILES: O=c1c(CNCc2cc(Br)ccn2)cc2ccc(N3CCOCC3)cc2n1CCOc1ccccc1] | GRG-02-24 (also referred to as VBT-2-24 and compound 2-24) | Chemical Formula: C₂₈H₂₉BrN₄O3 Exact Mass: 548.142 |
| 24 | 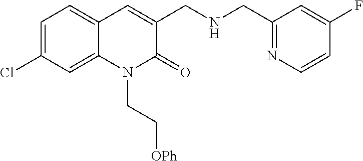 [SMILES: O=c1c(CNCc2cc(F)ccn2)cc2ccc(Cl)cc2n1CCOc1ccccc1] | GRG-03-30 (also referred to as VBT-3-30 and compound 3-30) | Chemical Formula: C₂₄H₂₁ClFN₃O₂ Exact Mass: 437.131 |
| 25 | 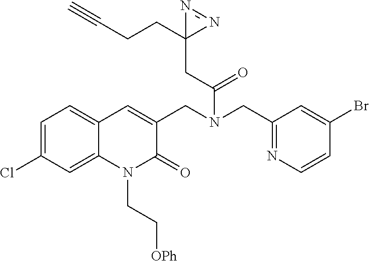 [SMILES: C#CCCC1(CC(=O)N(Cc2cc(Br)ccn2)Cc2cc3ccc(Cl)cc3n(CCOc3ccccc3)c2=O)N=N1] | GRG-02-174 (also referred to as VBT-2-174 and compound 2-174) | Chemical Formula: C₃₁H₂₇BrClN₅O₃ Exact Mass: 631.099 |
| 26 | 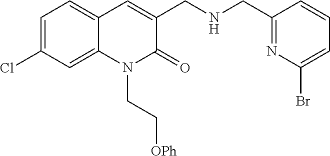 [SMILES: O=c1c(CNCc2cccc(Br)n2)cc2ccc(Cl)cc2n1CCOc1ccccc1] | ZH-2-44 (also referred to as VBT-2-44 and compound 2-44) | Chemical Formula: C₂₄H₂₁BrClN₃O₂ Exact Mass: 497.051 |
| 27 | 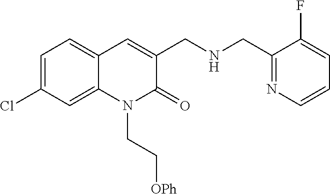 [SMILES: O=c1c(CNCc2ncccc2F)cc2ccc(Cl)cc2n1CCOc1ccccc1] | ZH-2-50 (also referred to as VBT-2-50 and compound 2-50) | Chemical Formula: C₂₄H₂₁ClFN₃O₂ Exact Mass: 437.131 |

### Example 11: Characterization Data for Representative Compounds

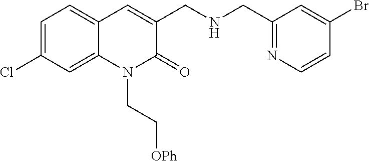 [SMILES: O=c1c(CNCc2cc(Br)ccn2)cc2ccc(Cl)cc2n1CCOc1ccccc1]

3-((((4-bromopyridin-2-yl)methyl)amino)methyl)-7-chloro-1-(2-phenoxyethyl) quinolin-2 (1H)-one (GRG-1-34/VBT-1-34): 7-chloro-2-oxo-1-(2-phenoxyethyl)-1,2-dihydroquinoline-3-carbaldehyde (50 mg, 0.15 mmol) was dissolved in dry dichloroethane (10 mL) to which was added (4-Bromopyridin-2-yl) methanamine (29 mg, 0.15 mmol) followed by the addition of Sodium triacetoxyborohydride (81 mg, 0.38 mmol) in the presence of catalytic amount of acetic acid. The reaction mixture was stirred overnight at room temperature under a nitrogen atmosphere to get crude product after aqueous workup. Pure GRG-1-34 (46 mg, 0.092 mmol) was obtained as a yellowish sticky solid after separation using prep HPLC (30% ACN in 0.5% TFA). ¹H NMR (500 MHZ, CDCl₃) θ 8.35 (d, J=5.3 Hz, 1H), 7.76-7.69 (m, 2H), 7.59 (d, J=1.9 Hz, 1H), 7.47 (d, J=8.2 Hz, 1H), 7.32 (dd, J=5.3, 1.9 Hz, 1H), 7.29-7.23 (m, 2H), 7.21 (dd, J=8.3, 1.8 Hz, 1H), 6.97-6.90 (m, 1H), 6.89-6.82 (m, 2H), 4.66 (t, J=5.7 Hz, 2H), 4.35 (t, J=5.6 Hz, 2H), 3.96 (s, 2H), 3.83 (d, J=1.2 Hz, 2H); ¹³C NMR (126 MHZ, CDCl₃) δ 162.1, 161.4, 158.1, 150.0, 140.1, 136.0, 135.7, 133.4, 130.8, 129.6, 125.6, 125.4, 122.8, 121.2, 119.0, 115.2, 114.3, 65.4, 54.2, 49.2, 42.7; HRMS (ESI, MH⁺) calcd for C₂₄H₂₂BrClN₃O₂: 498.0583, found 498.0584.

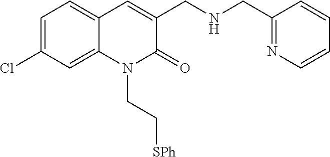

7-chloro-1-(2-(phenylthio)ethyl)-3-(((pyridin-2-ylmethyl)amino)methyl)quinolin-2 (1H)-one (GRG-1-104): 7-chloro-2-oxo-1-(2-(phenylthio)ethyl)-1,2-dihydroquinoline-3-carbaldehyde (48 mg, 0.14 mmol) was dissolved in dry dichloroethane (10 mL) to which was added pyridin-2-ylmethanamine (108 μl, 0.153 mmol) followed by the addition of Sodium triacetoxyborohydride (74 mg, 0.35 mmol) in the presence of catalytic amount of acetic acid. The reaction mixture was stirred overnight at room temperature under a nitrogen atmosphere to get crude product after aqueous workup. Pure GRG-1-104 (29 mg, 0.067 mmol) was obtained as a dirty white solid after separation using prep HPLC (30% ACN in 0.5% TFA). ¹H NMR (500 MHZ, CDCl₃) δ 8.44 (ddd, J=4.9, 1.8, 1.0 Hz, 1H), 7.58 (s, ¹H), 7.53 (td, J=7.7, 1.8 Hz, 1H), 7.42-7.36 (m, 2H), 7.34 (d, J=8.3 Hz, 1H), 7.29-7.20 (m, 2H), 7.18-7.11 (m, ¹H), 7.04 (ddd, J=7.6, 3.6, 1.5 Hz, 2H), 6.96 (d, J=1.8 Hz, 1H), 4.31-4.24 (m, 2H), 3.87 (s, 2H), 3.71 (d, J=1.3 Hz, 2H), 3.13-3.06 (m, 2H), 2.71 (s, ¹H); ¹³C NMR (126 MHZ, CDCl₃) δ 161.7, 159.4, 149.3, 139.0, 136.5, 136.1, 135.3, 134.6, 131.2, 130.1, 129.9, 129.3, 127.0, 122.7, 122.3, 122.1, 119.2, 113.6, 54.6, 49.0, 42.6, 30.3; HRMS (ESI, MH⁺) calcd for C₂₄H₂₃ClN₃OS: 436.1250, found 436.1245.

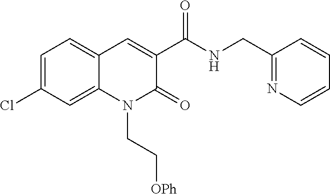 [SMILES: O=C(NCc1ccccn1)c1cc2ccc(Cl)cc2n(CCOc2ccccc2)c1=O]

7-chloro-2-oxo-1-(2-phenoxyethyl)-N-(pyridin-2-ylmethyl)-1,2-dihydroquinoline-3-carboxamide (GRG-1-75): 7-chloro-2-oxo-1-(2-phenoxyethyl)-1,2-dihydroquinoline-3-carbaldehyde (50 mg, 0.15 mmol) was subjected to Pinnick oxidation to get the oxidized acid as crude product, which was taken to second step without further purification and dissolved in dry DCM (10 mL) to which was added pyridin-2-ylmethanamine (19 μl, 0.183 mmol) followed by the addition of EDC·HCl (36 mg, 0.183 mmol. The reaction mixture was stirred for 3 hours at room temperature under a nitrogen atmosphere to get crude product after aqueous workup. Pure GRG-1-75 (23 mg, 0.053 mmol) was obtained as a dirty white solid after flash chromatography (SiO₂, 50% EtOAc in Hexane). ¹H NMR (500 MHZ, CDCl₃) δ 10.43 (t, J=6.0 Hz, 1H), 8.93 (s, ¹H), 8.64 (ddd, J=4.9, 1.8, 0.9 Hz, 1H), 7.85 (d, J=1.8 Hz, 1H), 7.73-7.64 (m, 2H), 7.39-7.34 (m, ¹H), 7.31 (dd, J=8.4, 1.8 Hz, 1H), 7.29-7.24 (m, 2H), 7.20 (ddd, J=7.5, 4.9, 1.2 Hz, 1H), 6.95 (d, J=1.1 Hz, 1H), 6.88-6.81 (m, 2H), 4.85 (d, J=5.4 Hz, 2H), 4.75 (t, J=5.5 Hz, 2H), 4.41 (t, J=5.4 Hz, 2H); ¹³C NMR (101 MHZ, DMSO) δ 158.5, 157.2, 153.2, 152.5, 144.7, 139.0, 136.9, 134.3, 131.9, 127.0, 124.8, 119.0, 117.4, 117.0, 116.6, 113.6, 111.0, 109.5, 60.7, 40.6, 38.5; HRMS (ESI, MNa⁺) calcd for C₂₄H₂₀ClN₃O₃: 456.1090, found 456.1085.

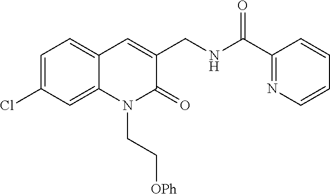 [SMILES: O=C(NCc1cc2ccc(Cl)cc2n(CCOc2ccccc2)c1=O)c1ccccn1]

N-((7-chloro-2-oxo-1-(2-phenoxyethyl)-1,2-dihydroquinolin-3-yl)methyl) picolinamide (GRG-1-94): 7-chloro-3-(hydroxymethyl)-1-(2-phenoxyethyl) quinolin-2 (¹H)-one (104 mg, 0.313 mmol), was dissolved in DCM (4 mL) and the reaction flask was placed in ice bath condition. To this solution was added PBr₃ (1 ml, 0.57 mmol) slowly, followed by the addition of 4 drops of Pyridine. The reaction mixture was slowly brought to room temperature and stirred for 1 hour. The solvent was removed and worked up to get a crude mixture, which was further dissolved in ethanol solution followed by the addition of ammonia water. This reaction mixture was stirred at room temperature overnight, the solvent was removed and dried. The intermediate was taken to the next step without purification. This was dissolved in DCM to which picolinic acid (15 mg, 0.117 mmol) and EDC·HCl (42 mg, 0.213 mmol) were added and stirred for 4 hours to get the crude product. Pure product GRG-1-94 (8 mg, 0.018 mmol) was obtained after flash chromatography (SiO₂, 50% EtOAc in Hexane) as a dirty white solid. ¹H NMR (500 MHZ, CDCl₃) δ 8.74 (t, J=6.5 Hz, 1H), 8.56 (ddd, J=4.8, 1.8, 0.9 Hz, 1H), 8.17 (dt, J=7.9, 1.1 Hz, ¹H), 7.82 (td, J=7.7, 1.7 Hz, 1H), 7.75 (s, ¹H), 7.71 (d, J=1.8 Hz, 1H), 7.44 (d, J=8.4 Hz, 1H), 7.40 (ddd, J=7.6, 4.7, 1.2 Hz, 1H), 7.26-7.19 (m, 2H), 7.17 (dd, J=8.4, 1.8 Hz, 1H), 6.91 (tt, J=7.3, 1.1 Hz, 1H), 6.88-6.79 (m, 2H), 4.67 (t, J=5.6 Hz, 2H), 4.62 (dd, J=6.6, 1.0 Hz, 2H), 4.35 (t, J=5.6 Hz, 2H); ¹³C NMR (126 MHZ, CDCl₃) δ 164.6, 162.1, 158.1, 149.8, 148.3, 140.3, 137.3, 136.3, 136.3, 129.9, 129.5, 129.2, 126.2, 122.9, 122.3, 121.2, 118.9, 115.2, 114.3, 65.4, 42.7, 39.7; HRMS (ESI, MNa⁺) calcd for C₂₄H₂₀ClN₃O₃: 456.1090, found 456.1084.

### Example 12: Activity of the Disclosed Compounds

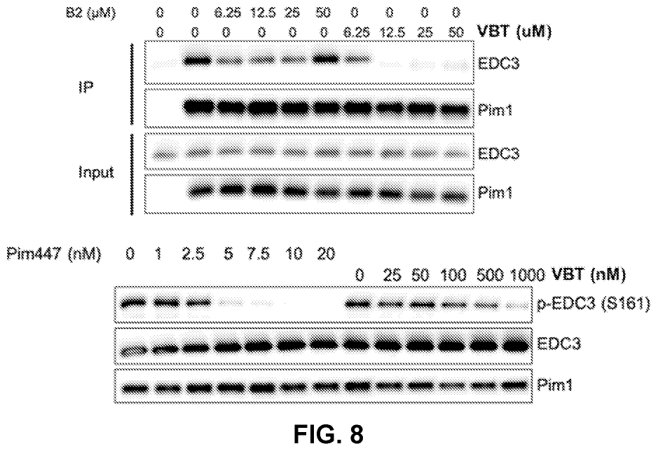

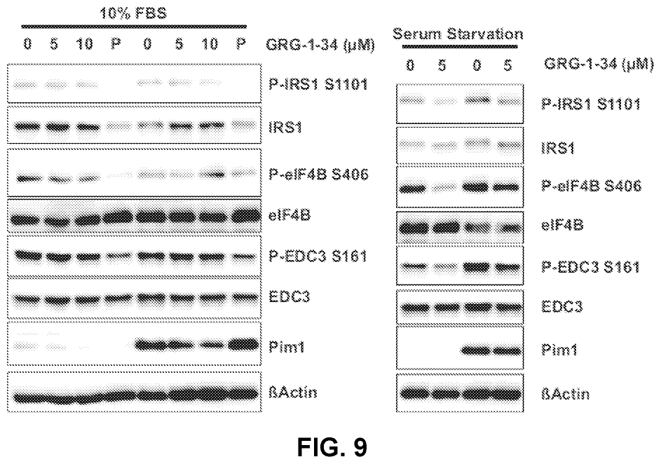

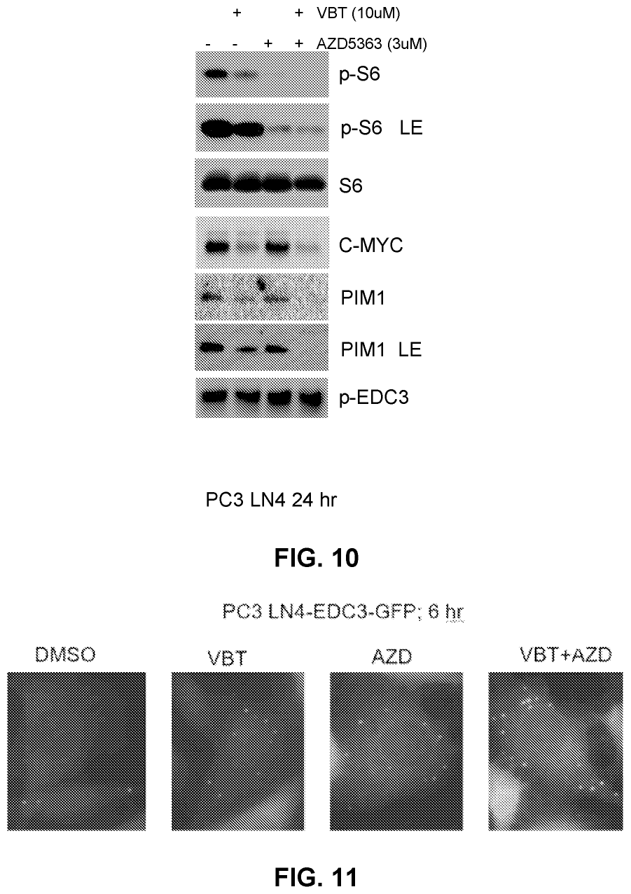

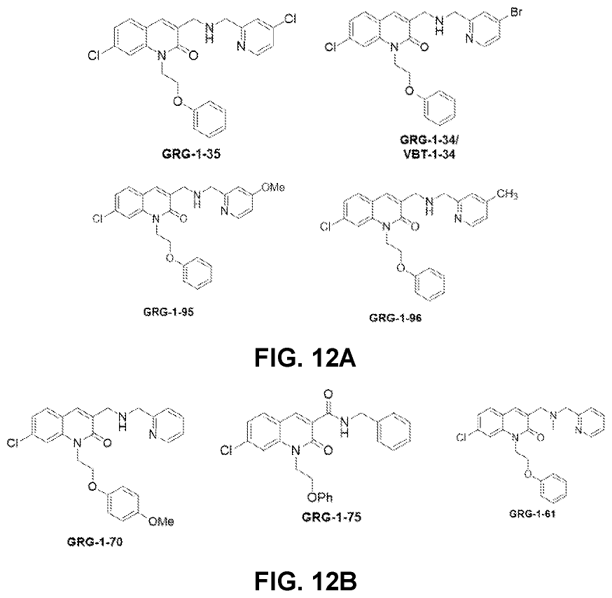

In vitro and in vivo testing of the disclosed compounds was carried out. VBT 1-34 blocks co-immunoprecipitation of EDC3 with Pim, suggesting that VBT 1-34 binds to the substrate binding site of Pim (FIG. 8). Application of VBT 1-34 to prostate cancer cells in culture also inhibits Pim activity (FIG. 9). Treatment of prostate cancer cell lines PC3-LN4 with VBT 1-34 decreases cellular levels of Pim1, c-Myc, and phosphorylation of the S6 protein (FIG. 10). Furthermore, addition of VBT 1-34 to prostate cancer cells increases the number of P-bodies in the cells; addition of AKT inhibitor AZD 5363 further increases P-bodies (FIG. 11). FIGS. 12A-12B and 13A-13B show the structures of some of the disclosed compounds tested herein.

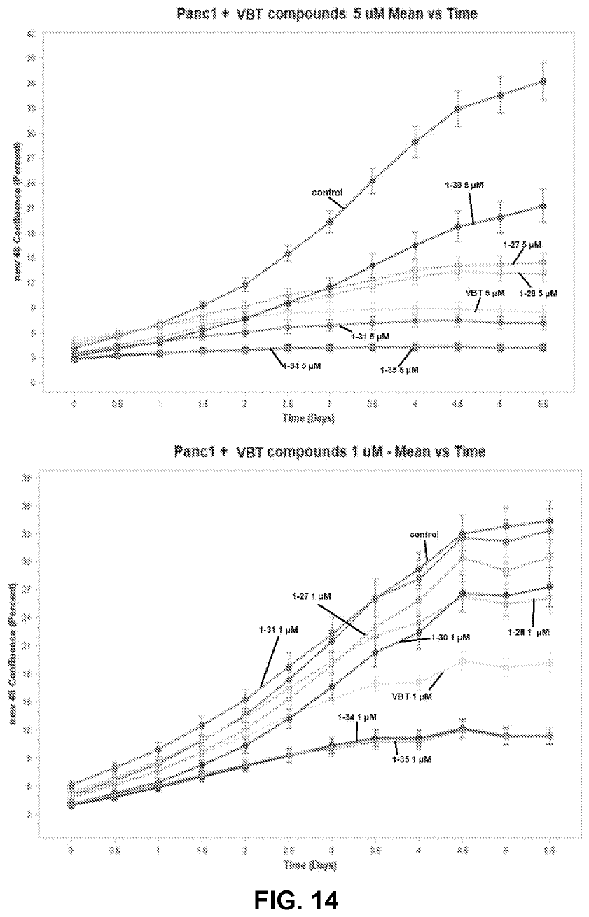

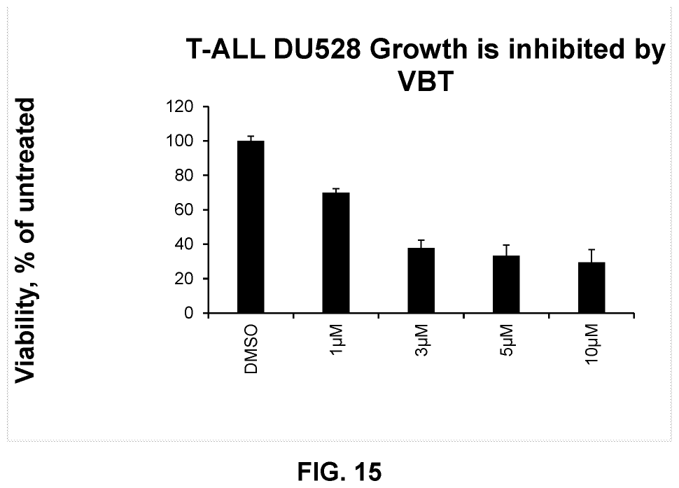

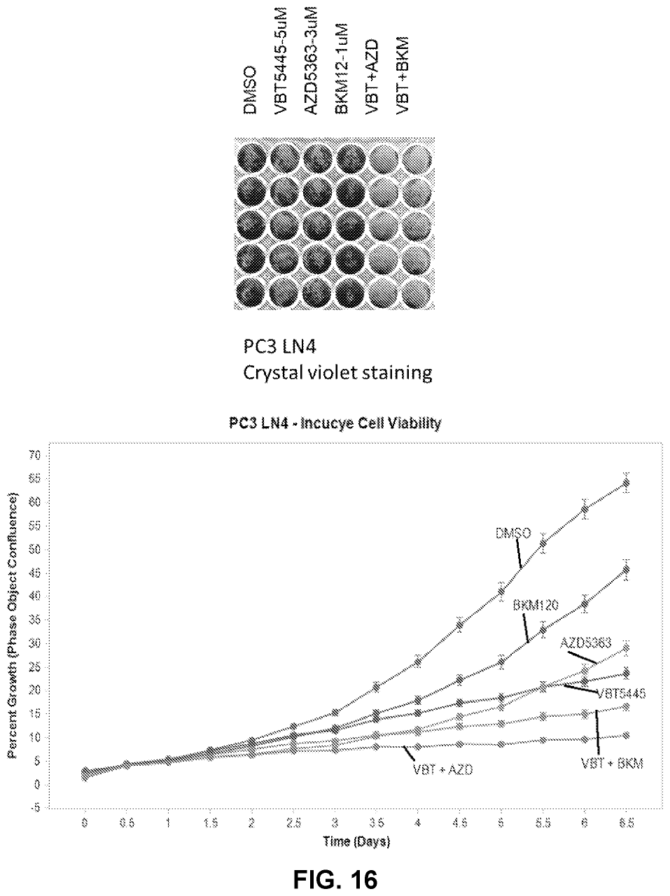

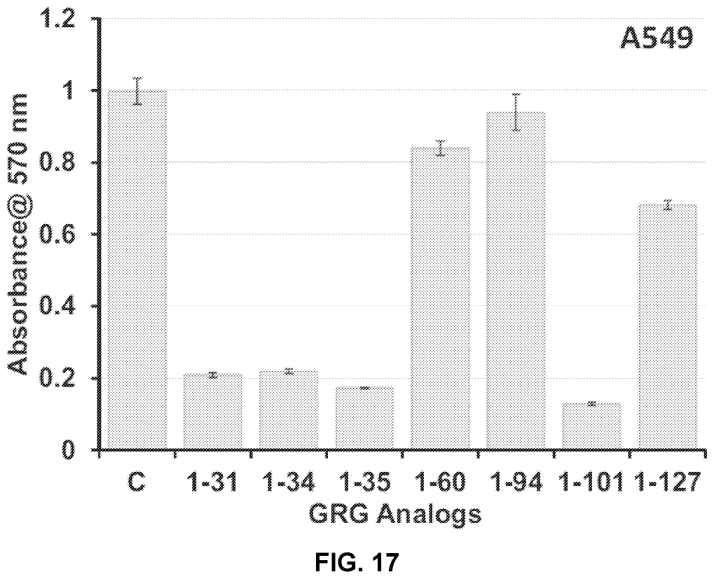

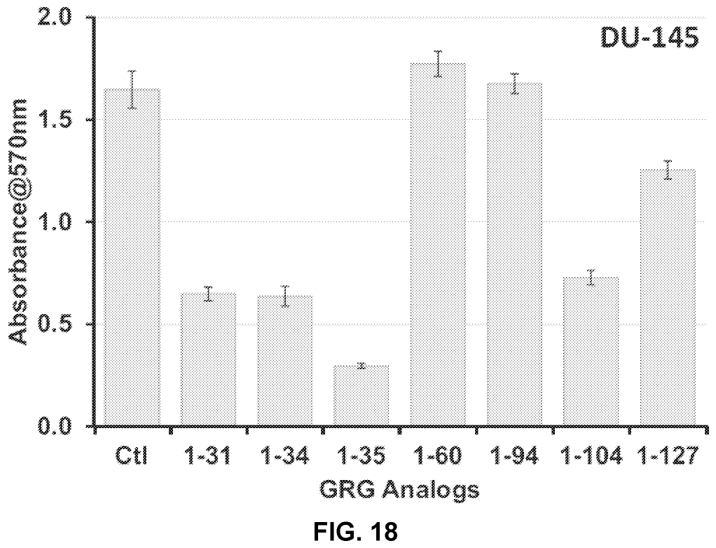

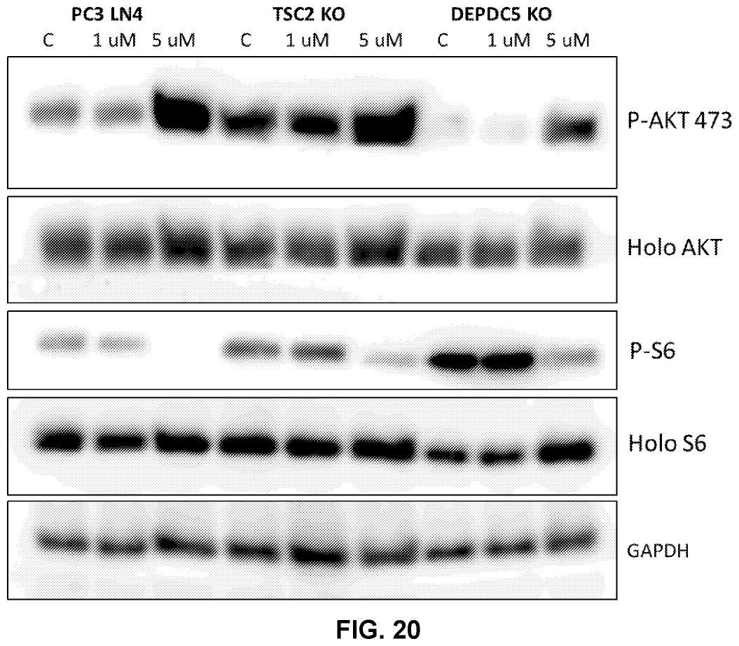

VBT 1-34 shows activity against pancreatic cancer cells (FIG. 14), T-acute lymphoblastic leukemia (FIG. 15), prostate cancer (FIG. 18), and lung cancer (FIG. 17). Activity against prostate cancer growth is enhanced by the addition of AKT/PI3K inhibitors AZD 5363 and BKM120 (FIG. 16). VBT 1-34 and related compounds also inhibit phosphorylation of mTORC substrates (FIGS. 19-20) and can be administered orally, peaking at a level with activity in cell culture (FIG. 2I).

Structure Activity Relationships: SAR relationships of the disclosed compounds are presented in Table 2:

TABLE 2
SAR Studies using Inhibition of Prostate Cancer Growth as a Readout

| Substituent Position | Activity | Example Compounds |
|---|---|---|
| 4-substituted compound | 6 active compounds | GRG-1-45, GRG-1-34, |
| | | GRG-1-95, GRG-1-96 |
| 5-substituted compound | 2 active compounds | GRG-1-27, GRG-1-28 |
| 6-substituted compound | 1 active compound (methoxy) | GRG-1-30, ZH-2-44 |
| | and 1 less active compound | |
| | (hydroxy) | |
| 3-substituted compound | Less active | ZH-2-50 |
| p-methoxy substituent in | Brings activity, though 4-chloro | GRG-1-70, GRG-1-127, |
| phenyl ring | substitution decreases activity | GRG-1-166, GRG-1-167 |
| Thiophenyl compound | Active | GRG-1-104 |
| Replacement of amine | Reduces activity to some extent | GRG-1-61 |
| proton | | |
| Replacement of nitrogen with | Contributes to loss of activity | GRG-1-60, GRG-1-63 |
| oxygen | | |
| Introduction of carbonyl | To be determined | GRG-1-75 |
| group close to pyridine | | |
| Morpholine substitution in | Contributes to loss of activity | GRG-02-24 |
| place of chlorine in quinoline | | |

### Example 13: Biological Materials and Methods

Antibodies and Reagents: The following antibodies were purchased from Cell Signaling Technology anti-Pim1 (Cat #2907), anti-S6 (Cat #9202), anti-phopho-S6 (Ser 235/236 Cat #4856), anti-EDC3 (Cat #14495), anti-phospho-GSK3B, (Ser9, Cat #9336), anti-phospho-IRS1 S1101 (Cat #2385), anti-phospho-elF4B S406 (Cat #5399), anti-elF4B (Cat #3592), anti-AKT (Cat #9272), and anti-phospho-AKT S473 (Cat #4058), anti-phospho 4E-BP1 (T37/46) (Cat #2855), anti-4E-BP1 (Cat #9644), c-Myc (Cat #9402). The anti-phospho-EDC3 (S161 Cat #600-401-J38) was purchased from Rockland Antibodies while the HRP conjugated anti-β-actin (Cat #A3854) was from Sigma. Anti-IRS1 (Cat #06-248) was obtained from Merck Millipore. Antibodies bought from Santa Cruz Biotechnology included anti-EDC3 (Cat #sc-365024) and anti-GAPDH HRP (Cat. #0411). The kinase inhibitors BKM 120 (Cat #A11016) and AZD5363 (Cat #A11759) were purchased from Adooq Bioscience. VBT5445 was purchased from Chembridge.

Cell culture: The prostate cancer cell lines PC3-LN4, DU-145 and LNCaP, lung cancer cell line A⁵⁴⁹ and breast cancer MDA-MB-231 cells were purchased from the American Type Culture Collection (ATCC). T-cell acute lymphoblastic leukemia cell line DU.528 was a kind gift of Jon C. Aster (Harvard Medical School, Boston, MA). The PANC1 cell line was a gift of Dr. N. Lee (University of Arizona School of Medicine, Tucson, AZ). These cell lines were cultured in RPMI with 10% fetal bovine serum (FBS) and 1% Penicillin and Streptomycin at 37° C. in 5% CO2.

Immunofluorescence: Immunofluorescence (IF) staining was performed on PC3-LN4 cells stably expressing EDC3-GFP. 1×10⁴ cells were seeded in each chamber of an 8 well chamber slide (Nunc Lab-Tek II Chamber Slide System, Life Technologies) and cultured for 48 h. After attaching to the chamber slides cells were treated with DMSO, VB-5445 (5 M), AZD5363 (3 μM), or the combination for 6 h. Cells were then fixed with 4% paraformaldehyde in PBS for 15 min and subsequently permeabilized with 0.2% Triton X-100 in PBS. Cells were stained with diluted DAPI (1:4000) in 1% nor-mal goat serum in PBS. Cells were mounted with Prolong Gold Antifade mounting medium (Molecular Probes).

Cell growth assay: Tumor cells were seeded into 48 well plates (1000 cells/well) and treated with varied agents. The Incucyte real-time imaging system (Incucyte) was used to measure cell proliferation using a non-label cell monolayer confluence approach. The images were captured every 12 h. Graphs were generated using an integrated confluence metric as an alternative for cell numbers. At the end of the study cell growth was visualized by crystal violet staining.

Immunoblotting: Western blots were performed as described previously. Briefly, protein lysates (30 μg) were subjected to SDS-PAGE and transferred to nitrocellulose membranes. After blocking with 5% milk in TBST buffer for 30 min at room temperature, the membranes were incubated overnight at 4° C. with the Indicated primary antibody in 3% BSA in TBST. Specific proteins were detected with the corresponding HRP conjugated secondary antibody using enhanced chemiluminescence (GE Lifesciences, Piscataway, NJ).

Cell viability (XTT) assay: T-ALL cells were seeded into 96 well plates at a density of 20,000 cells per well and treated with VBT-5445 for 72 h, using DMSO as control. Cell viability was measured using XTT cell proliferation assay (Trevigen Cat #4891-025-K) following the manufacturer's protocol. Briefly, XTT reagent was added to cell culture after the treatment and incubated for 4 h at 37° C. and 5% CO₂. The absorbance of the colored formazan product was measured at 450 nm.

Plasma concentrations of GRG-1-34: These concentrations were determined by high performance liquid chromatography (HPLC) tandem mass spectrometry (MS). Quantification was achieved by GRG-1-34 calibration standards prepared in mouse plasma. GRG-1-34 in calibration standards or authentic samples was extracted by protein precipitation with methanol. After centrifugation, the supernatant was injected onto the HPLC-MS system. HPLC separation was achieved using a C-18 column and a gradient mobile phase of water and acetonitrile, both with 0.1% formic acid. The mass spectrometer was operated in the electrospray positive ion mode with selective reaction monitoring. The calibration curve was linear over the plasma concentration range of 2-1800 ng/ml. The pharmacokinetic analysis was performed using PKSolver 2.0 by a noncompartmental analysis. Murine gavage with GRG 1-34 was approved by the Animal Care Committee of the University of Arizona.

### Example 14: Results of Biological Studies

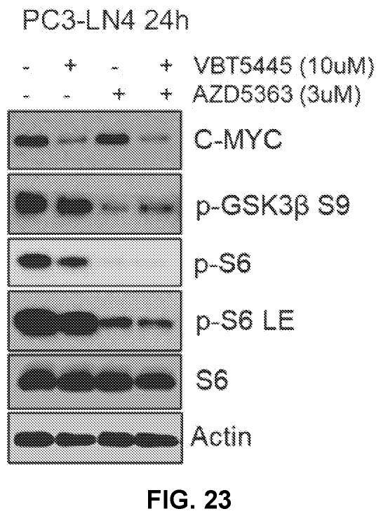

Computer screening followed by surface plasmon resonance (SPR) yielded a lead compound VBT-5445 that can interact with the Pim-1 protein kinase. Enhancer of mRNA Decapping 3 (EDC3) is a substrate of the Pim kinase that regulates the decapping of messenger RNA, and its activity is modulated by Pim mediated phosphorylation. To evaluate the ability of VBT-5445 to inhibit phosphorylation of EDC3 on serine 161 by Pim1, a kinase assay was carried out with purified Pim1 and EDC3 (FIG. 7B). At the concentration of 1 μM, this compound inhibited EDC3 phosphorylation; and at higher concentrations, this molecule blocked the ability of Pim1 to coimmunoprecipitate with EDC3 (FIG. 7B). Agents capable of inhibiting Pim protein kinase induce P-bodies in tumor cells. P-bodies are RNA and protein granules that store and degrade messenger RNAs that form under specific stress stimuli. It was found that the addition of VBT5445 for 6 h induces increased numbers of P-bodies and works in concert with an AKT kinase inhibitor, AZD5363, to further increase P-body formation (FIG. 11). Based on these results, the growth inhibitory activity of this compound was tested either alone or in combination with AZD 5363 or BKM120, a PI3 Kinase inhibitor, in prostate cancer PC3-LN4 cells. As demonstrated for P-bodies, the combination of these agents is more active in inhibiting cancer growth (FIG. 16). In addition, VBT5445 inhibited the growth of the T-cell acute lymphoblastic leukemia cell line DU528 in a dose-dependent fashion (FIG. 15). Inhibitors of the Pim protein kinase are known to block mTORC protein kinase activity and to inhibit the cellular levels of the c-Myc proto-oncogene. When applied to PC3-LN4 prostate cancer cells VBT-5445 blocked mTORC1 activity based on a decrease in the phosphorylation of the S6 protein and the levels of c-Myc protein and worked in concert with the AKT inhibitor (FIG. 23). These initial results suggested that this compound had broad growth inhibitory activity and regulated important cellular pathways controlling RNA storage and decapping, and protein translation.

Because of ease of growth, PANC1 pancreatic cancer cells were used to test the activity of derivatives of VBT-5445 (FIG. 14). The com-pound's ability to inhibit growth was suggested to be dose-dependent (FIG. 14), with 1 μM dosing having less growth inhibitory activity (FIG. 14). The most active com-pounds in this series were GRG1-31, 1-34, 1-35, and 1-104 while others, for example, 1-60 and 1-94 were inactive. GRG-1-34 treatment of prostate cancer cells in culture demonstrated that in serum-starved conditions treatment decreased Pim substrate phosphorylation including IRS1, elF4B, and EDC3. Using a doxycycline inducible Pim kinase vector in these tumor cells, this inhibition of phosphorylation was proportional to the level of Pim in these cells (FIG. 9). Testing the growth inhibitory activity of these specific chemicals against the lung cancer cell line A549 and the prostate cancer cell line DU-145 yielded a similar activity profile (FIGS. 17-18). Overnight incubation of PANC1 pancreatic cancer cells with these GRGs demonstrated that the growth inhibitory activity of these chemicals paralleled their ability to decrease mTORC kinase activity. This is demonstrated by the ability of GRG-1-31, 1-34, 1-35 and 1-104 but not 1-60 and 1-94 to block the phosphorylation of mTORC substrates including AKT and 4E-BP1 and the phosphorylation of ribosomal S6 protein (S6), an indicator of mTORC1 activation (FIG. 19). In the prostate cancer cell line PC3-LN4 (FIG. 20, lanes 1-3) treatment with GRG-1-34 inhibited the phosphorylation of the mTORC1 substrate S6 protein but increased the phosphorylation of AKT on serine 473. Inhibition of mTORC1 is known to stimulate the feedback activation of mTORC2 leading to AKT phosphorylation. This biochemical activity of the GRGs appeared to be context-dependent as DU-145 cells do not evidence this feedback while the breast cancer MDA-MB231 cells do. The effect of these compounds on mTORC1 and 2 was still evident in PC3-LN4 cells that contained a knockout (KO) using CRISPR/CAS9 of either TSC2 or DEPDC5 (FIG. 12, lanes 4-9). TSC2 KO activated the Rheb GTPase while DEPDC5 KO activated the RAG GTPase both of which stimulate mTORC1 activity. Thus, GRG compounds were capable of inhibiting mTORC1 activity even when this kinase was highly activated.

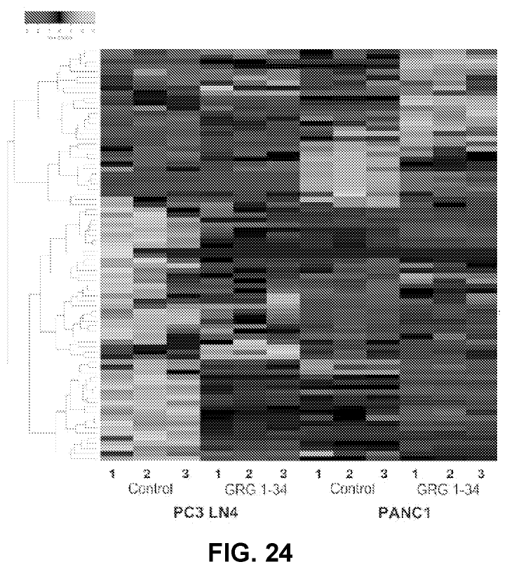

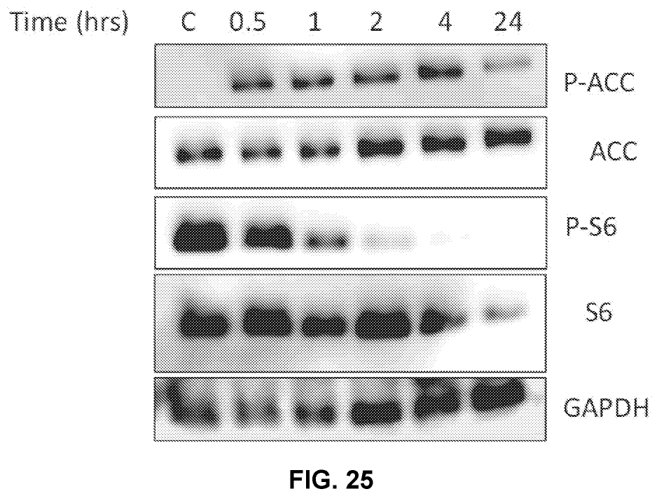

To further examine how these compounds function to inhibit tumor growth, changes in both a wide variety of phosphoprotein and protein levels using reverse-phase protein arrays (RPPA) were evaluated. PANC1 and PC3-LN4 cells were treated with GRG1-34 at 5 μM for 24 h. For controls, the PANC1 cells were incubated with either pp242, an mTORC inhibitor, or Pim447, a Pim inhibitor for 6 h. The values in the RPPA obtained at these two time points were divided by the value at time zero to allow an evaluation of the changes caused by each drug treatment. The data demonstrated that (1) the response of the prostate versus pancreatic cancer cell lines to GRG-1-34 treatment while similar was not identical as seen in the heat map (FIG. 24), and (2) the effect of GRG-1-34 on the results of the RPPA was not identical to either the effect of known mTORC or Pim inhibitors (Table 3) suggesting that the GRG compounds had different activity from either of these drugs and thus are novel in their function. The changes in the mTORC signaling pathway documented by RPPA (FIG. 24) showed inhibition by this compound of mTORC, e.g., phosphorylations of mTOR S2448, 40 S ribosomal protein S6 S235/235, S6K T389, 4E-BP1 S65. Additionally, on RPPA the phosphorylation of AMPK T172 and acetyl COA carboxylase S79 was increased suggesting that the AMPK activation pathway could be important in inhibiting mTOR activity. As documented in the LNCaP prostate cancer cell line (FIG. 25) GRG-1-35, which has similar activity to GRG-1-34, treatment increased the phosphorylation of acetyl CoA carboxylase (as shown on Western blotting in PC3-LN4) consistent with the results seen on RPPA.

TABLE 3
Representative Protein Changes on RPPA after pp242,
Pim447, or GRG-1-34 Treatment of PANC1 Cells for 6 h^{a}

| | 6hRx | 6hRx | 6hRX |
|---|---|---|---|
| | pp242/0 | Pim447/0 | GRG1-34/0 |
| Treatment | time | time | time |
| 4EBP1S65 | 0.22 | 0.42 | 0.52 |
| ERKT202_Y204 | 2.04 | 0.85 | 2.39 |
| FKHR-FOX01S256 | 1.03 | 1.45 | 0.71 |
| GSK3a_bS21_9 | 1.09 | 0.66 | 1.66 |
| p70S6KT389 | 0.29 | 0.39 | 0.74 |
| S6RibosomalPS235_236 | 0.34 | 0.32 | 1.05 |
| ATMS1981 | 0.46 | 1 | 0.46 |
| cAbIY245 | 1.54 | 1.77 | 0.87 |
| CICaspase3D175 | 0.7 | 0.64 | 1.09 |
| CofilinS3 | 0.6 | 1.33 | 0.46 |
| FAKY576_577 | 0.81 | 1.03 | 0.78 |
| HistoneH2AXS139 | 1.19 | 1.56 | 0.76 |
| HistoneH3S10 | 0.64 | 0.93 | 0.69 |
| Jak1Y1022_1023 | 0.71 | 0.9 | 0.79 |
| MAPKpTEpY | 0.64 | 0.47 | 1.35 |
| PaxillinY118 | 0.9 | 0.84 | 1.08 |
| PDGFRbY751 | 0.67 | 0.1 | 7.02 |
| RetY905 | 0.25 | 0.32 | 0.77 |
| Stat3Y705 | 0.51 | 0.32 | 1.57 |
| Tyk2Y1054_1055 | 0.51 | 1.32 | 0.39 |
| VEGFR2Y1175 | 0.34 | 0.62 | 0.54 |
| YAPS127 | 0.76 | 1.11 | 0.68 |
| CrkIIY221 | 1.57 | 0.99 | 1.58 |
| PP1aT320 | 0.61 | 1.3 | 0.47 |
| p70S6KS371 | 0.39 | 0.63 | 0.62 |
| 4EBP1T70 | 0.32 | 0.42 | 0.75 |
| cKitY703 | 1.09 | 0.61 | 1.8 |
| p38MAPKT180_Y182 | 0.59 | 0.51 | 1.15 |
| p90RSKS380 | 0.91 | 0.6 | 1.51 |
| S6RibosomalProtein S240_244 | 0.66 | 0.7 | 0.95 |
| SAPK_JNKT183_Y185 | 1.07 | 1.25 | 0.85 |
| Stat2Y690 | 0.76 | 0.26 | 2.89 |
| IL-1bcleavedD116 | 0.46 | 1.14 | 0.41 |

^{a}The results of a 6-hr treatment values with pp242, Pim447, or GRG-1-34 addition were divided by the starting zero-time values. Each of these experiments was done in triplicate and the average values is listed.

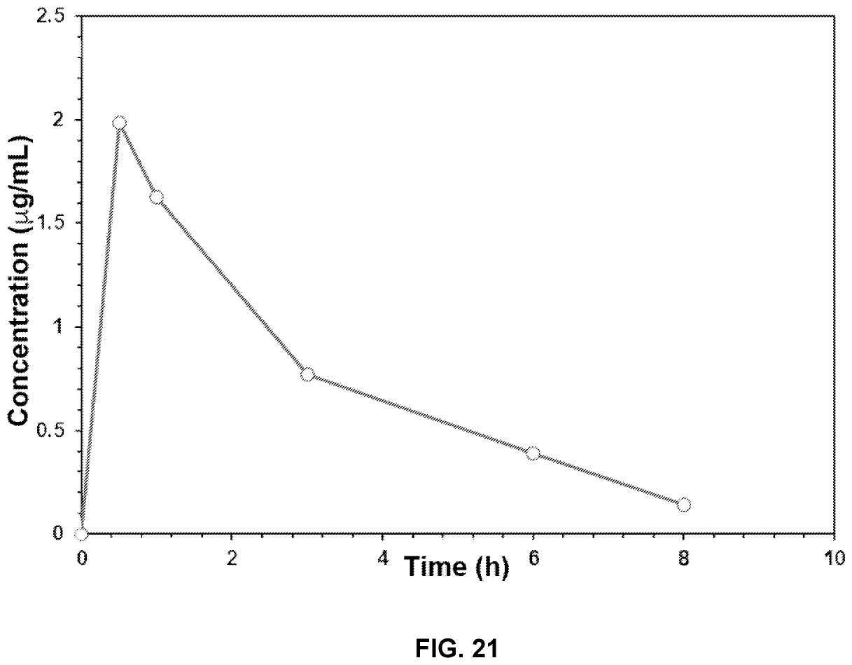

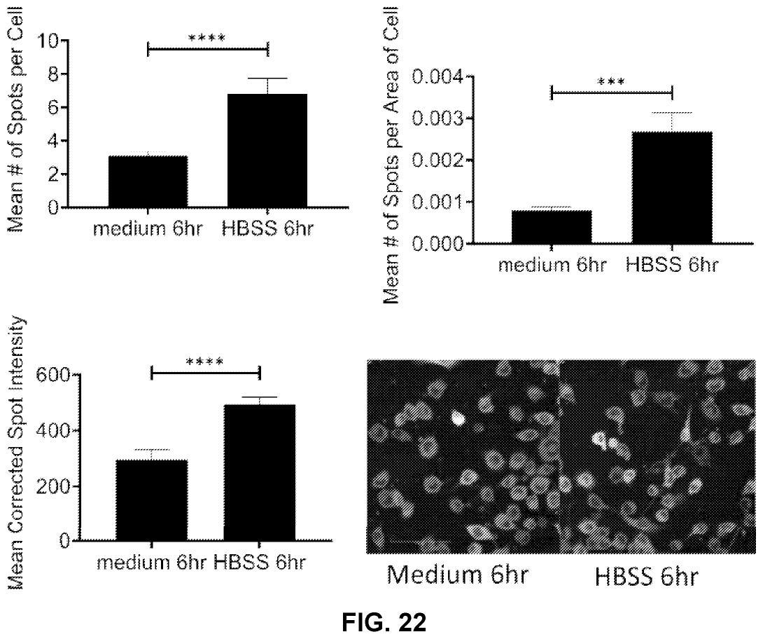

To examine the pharmacokinetics of this class of com-pounds GRG 1-34, one of the active isoforms, was given orally and blood was sampled from 3 mice at each time point (FIG. 21). The plasma concentration of GRG-1-34 peaked at 0.5 h after oral dosing and declined as a function of time. The average maximum plasma concentration (Cmax) was 1988 ng/ml following a dose of 62 mg/kg. The half-life (T_{1/2}) of GRG-1-34 was 2.077 h. It has a large apparent volume of distribution (VZ/F) and apparent systemic clearance (CL/F), likely attributed to low systemic bioavailability (F) after oral dosing. At the maximum blood concentration of 2 ng/ml, GRG-1-34 reached a concentration of 4 μM which in cell culture was sufficient to inhibit cell growth.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Aizer, A., et al. Journal of Cell Science 127, 4443-4456 (2014).
2. Anderson, P. et al. Journal of Cell Biology 172, 803-808 (2006).
3. Ayala, G., et al. Clin Cancer Res 10, 6572-6578 (2004).
4. Bailey, T. L., et al. Nucleic Acids Res 37, W202-208 (2009).
5. Balagopal, V. et al. Current Opinion in Cell Biology 21, 403-408 (2009).
6. Bandyopadhyay, S., et al. Cancer Res 64, 7655-7660 (2004).
7. Bearss, J. J., et al. EMBO Rep 22, e50835 (2021).
8. Bedolla, R., et al. Clin Cancer Res 13, 3860-3867 (2007).
9. Blewett, N., et al. RNA 17, 535-543 (2011).
10. Cai, D., et al. Trends Biochem Sci (2021).
11. Cen, B., et al. Mol Cell Biol 34, 2517-2532 (2014).
12. Chen, W. W., et al. Molecular Cancer Research 3, 443-451 (2005).
13. Clyman, R. I., et al. Circ Res 67, 175-186 (1990).
14. Comoglio, F., et al. BMC Bioinformatics 16, 32 (2015).
15. Conley-LaComb, M. K., et al. Mol Cancer 12, 85 (2013).
16. Cougot, N., et al. Journal of Cell Science 127, 4692-4701 (2014).
17. Crabb, S. J., et al. J Clin Oncol 39, 190-201 (2021).
18. Dai, H., et al. Prostate 65, 276-286 (2005).
19. de Bono, J. S., et al. Clin Cancer Res 25, 928-936 (2019).
20. Dolloff, N. G., et al. Oncogene 24, 6848-6854 (2005).
21. Engreitz, J. M., et al. Science 341, 1237973 (2013).
22. Eulalio, A. et al. Nature Reviews Molecular Cell Biology 8, 9-22 (2007).
23. Fernandes, N. et al. Nucleic Acids Res 48, 6265-6279 (2020).
24. Floor, S. N., et al. Nat Struct Mol Biol 17, 1096-1101 (2010).
25. Gunaratne, J., et al. J Biol Chem 286, 18093-18103 (2011).
26. Hafner, M., et al. Cell 141, 129-141 (2010).
27. Halstead, J. M., et al. Science 347, 1367-1671 (2015).
28. Hubstenberger, A., et al. Molecular Cell 68, 144-+ (2017).
29. Jonas, S. et al. Genes Dev 27, 2628-2641 (2013).
30. Khanna, R. et al. EMBO J 23, 1968-1976 (2004).
31. Licatalosi, D. D., et al. Nature 456, 464-469 (2008).
32. Ling, S. H. M., et al. Molecular and Cellular Biology 28, 5965-5976 (2008).
33. Liu, R. et al. J Virol 90, 7864-7879 (2016).
34. Lo, U. G., et al. Cancer Res 79, 1098-1112 (2019).
35. Lobel, J. H. et al. RNA 26, 1380-1388 (2020).
36. Lobel, J. H., et al. Proc Natl Acad Sci USA 116, 23512-23517 (2019).
37. Love, M. I. et al. Genome Biol 15, 550 (2014).
38 Luszczak, S., et al. Signal Transduction and Targeted Therapy 5 (2020).
39 Malik, S. N., et al. Clin Cancer Res 8, 1168-1171 (2002).
40. McCall, P., et al. Br J Cancer 98, 1094-1101 (2008).
41. McHugh, C. A., et al. Nature 521, 232-236 (2015).
42. McMenamin, M. E., et al. Cancer Res 59, 4291-4296 (1999).
43. Nagarajan, V. K., et al. Nucleic Acids Res 47, 9216-9230 (2019).
44. Nguyen, H. M., et al. Prostate 77, 654-671 (2017).
45. Padi, S. K. R., et al. Proc. Nat. Acad. Sci. USA 116, 20505-20510 (2019).
46. Paquette, D. R., et al. Nucleic Acids Res 46, 6318-6329 (2018).
47. Pound, C. R., et al. JAMA 281, 1591-1597 (1999).
48. Riggs, C. L., et al. J Cell Sci 133 (2020).
49. Rubenstein, C. S., et al. Cancer Res 79, 4703-4714 (2019).
50. Santio, N. M., et al. PLOS One 10, e0130340 (2015).
51. Schutz, S., et al. Nucleic Acids Research 45, 6911-6922 (2017).
52. Sheth, U. et al. Science 300, 805-808 (2003).
53. Song, J. H., et al. Oncotarget 7, 20152-20165 (2016).
54. Sroka, I. C., et al. J Cell Biochem 117, 491-499 (2016).
55. Tenekeci, U., et al. Mol Cell 62, 943-957 (2016).
56. Tibble, R. W., et al. Nat Chem Biol 17, 615-623 (2021).
57. Tritschler, F., et al. Molecular and Cellular Biology 27, 8600-8611 (2007).
58. Tritschler, F., et al. Mol Cell 33, 661-668 (2009).
59. Valdman, A., et al. Journal of Urology 171, 110-110 (2004).
60. Wang, Y., et al. Molecular Cell 60, 118-130 (2015).
61. Yu, X., et al. Plant Cell 28, 2385-2397 (2016).
62 Yu, X., et al. Dev Cell 56, 125-140.e126 (2021).

1. A compound having a structure of Formula I: 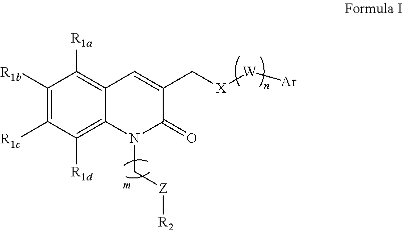
wherein each of R₁ₐ-R_{1d} is independently selected from hydrogen, halogen, or 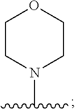
wherein m is from 1 to 6;
wherein Z is O, NH, NR₆, or S;
wherein R₂ is substituted or unsubstituted aryl or heteroaryl;
wherein R₆ is hydrogen, substituted or unsubstituted C₁-C₁₀ linear or branched alkyl, or substituted or unsubstituted alkyne;
wherein X is NR₃, O, or S;
wherein, when X is NR₃, R₃ is hydrogen, substituted or unsubstituted C₁-C₁₀ linear or branched alkyl, or substituted or unsubstituted alkyne;
wherein each occurrence of W is CH₂ or C(═O);
wherein n is from 0 to 4;
and wherein Ar is a substituted or unsubstituted C₁-C₁₀ aryl or heteroaryl group.

2. The compound of claim 1, wherein R₁ₐ, R_{1b}, and Ria are hydrogen and R_{1c} is halogen or

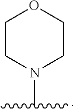 [SMILES: *N1CCOCC1]

3. The compound of claim 2, wherein R_{1c} is chlorine.

4. (canceled)

5. The compound of claim 1, wherein m is 2.

6. The compound of claim 1, wherein R₂ is

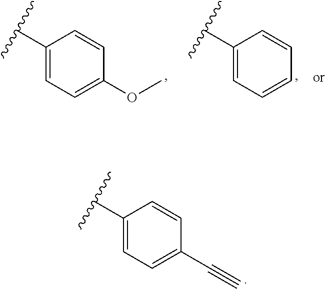

7.-9. (canceled)

10. The compound of claim 1, wherein X is O.

11. The compound of claim 1, wherein X is NR₃ and R₃ is H, methyl, or

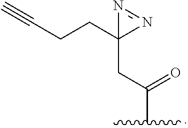 [SMILES: *C(=O)CC1(CCC#C)N=N1]

12.-13. (canceled)

14. The compound of claim 1, wherein n is 0 or 1.

15.-16. (canceled)

17. The compound of claim 1, wherein Ar is 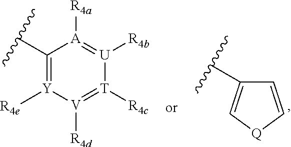
wherein A is C or N, wherein when A is N, R₄ₐ is absent;
wherein U is C or N, wherein when U is N, R_{4b} is absent;
wherein T is C or N, wherein when T is N, R_{4c} is absent;
wherein V is C or N, wherein when V is N, R_{4d} is absent;
wherein Y is C or N, wherein when Y is N, R₄ₑ is absent;
wherein R₄ₐ-R₄ₑ, if present, are independently selected from hydrogen, halogen, alkoxy, hydroxy, or substituted or unsubstituted C₁-C₄ alkyl, or wherein R_{4b} and R_{4c} together form a 5- or 6-membered aryl or heteroaryl ring;
wherein Q is selected from O, S, or NR₅, wherein R₅ comprises hydrogen or C₁-C₄ alkyl.

18. The compound of claim 1, wherein Ar is

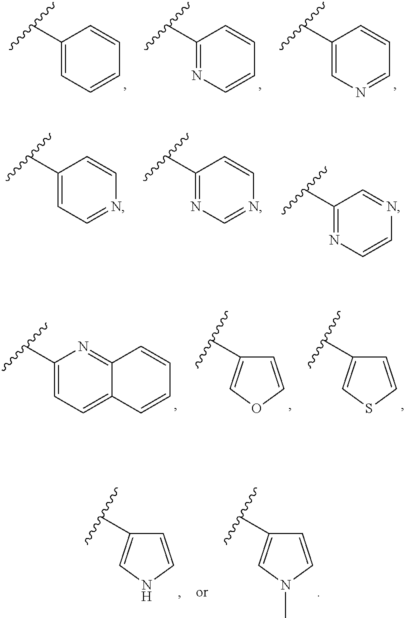

19.-20. (canceled)

21. The compound of claim 17, wherein each of R₄ₐ-R_{4d} is hydrogen.

22.-26. (canceled)

27. The compound of claim 1, wherein the compound is 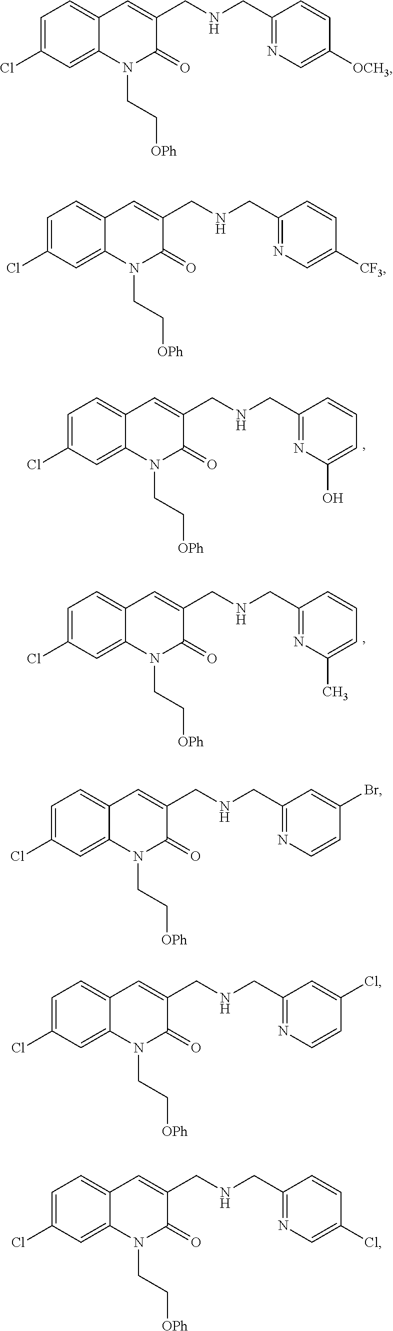 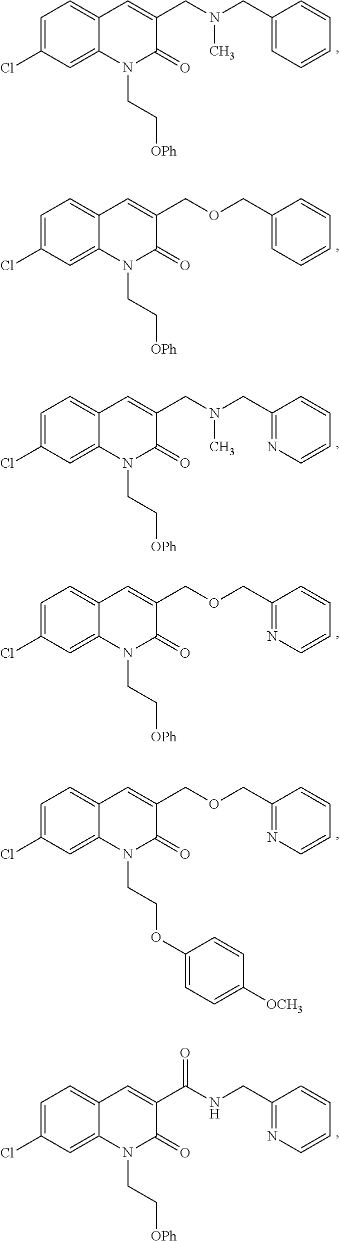 [SMILES: CN(Cc1ccccc1)Cc1cc2ccc(Cl)cc2n(CCOc2ccccc2)c1=O ; O=c1c(COCc2ccccc2)cc2ccc(Cl)cc2n1CCOc1ccccc1 ; CN(Cc1ccccn1)Cc1cc2ccc(Cl)cc2n(CCOc2ccccc2)c1=O ; O=c1c(COCc2ccccn2)cc2ccc(Cl)cc2n1CCOc1ccccc1 ; COc1ccc(OCCn2c(=O)c(COCc3ccccn3)cc3ccc(Cl)cc32)cc1 ; O=C(NCc1ccccn1)c1cc2ccc(Cl)cc2n(CCOc2ccccc2)c1=O] 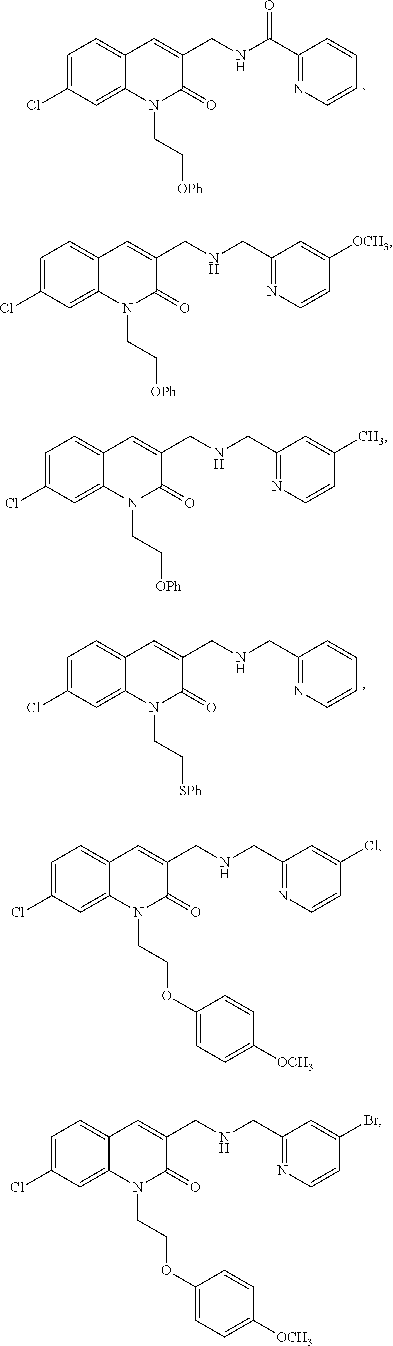 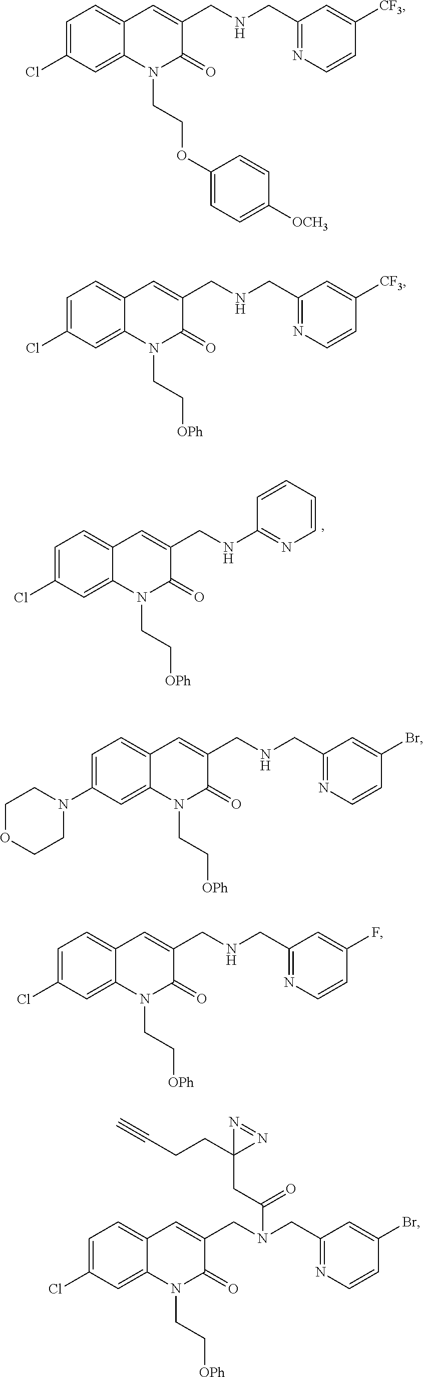 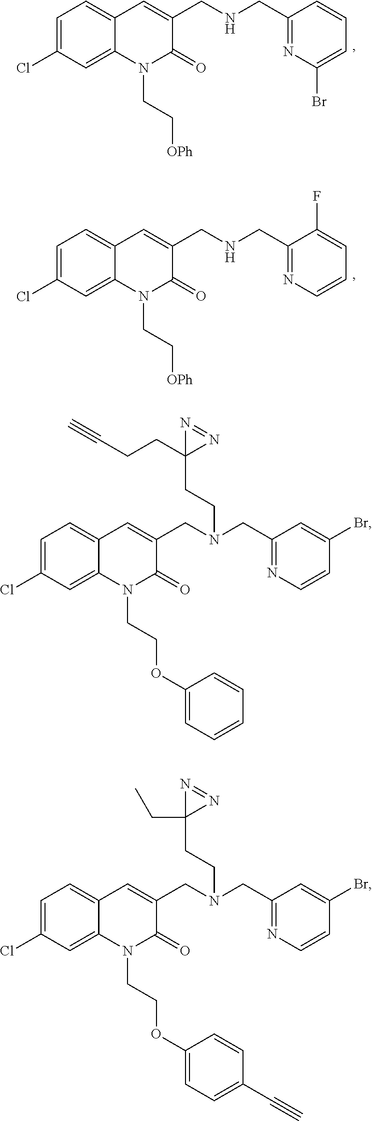 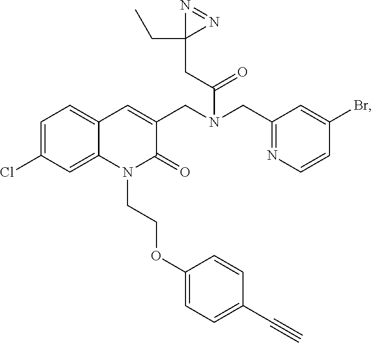 [SMILES: C#Cc1ccc(OCCn2c(=O)c(CN(Cc3cc(Br)ccn3)C(=O)CC3(CC)N=N3)cc3ccc(Cl)cc32)cc1]
or any combination thereof.

28. The compound of claim 1, wherein the compound is an allosteric Pim kinase inhibitor or an mTOR inhibitor

29. (canceled)

30. A pharmaceutical composition comprising the compound of claim 1 or a pharmaceutically acceptable salt thereof.

31. (canceled)

32. The pharmaceutical composition of claim 30, further comprising at least one AKT inhibitor, wherein the at least one AKT inhibitor comprises Akti-1/2, API-1, API-2, AT 7867, AZD 5363, 10-DEBC hydrochloride, FPA 124, GSK 690693, Perifosine, PHT 427, SC 66, KP372-1, AKTide-2T TFA, AKTide-2T, SC79, Honokiol, TD52, TASP0415914, ACT001, Artemisinin, Recilisib, Guggulsterone, Scutellarin, Triciribine, α-linolenic acid, miltefosine, deguelin, LM22B-10, 1,3-dicaffeoylquinic acid, pachymic acid, cenisertib, sophocarpine, esculetin, borussertib, Paris saponin VII, amicolide D, N-oleoyl glycine, CHPG, hematein, loureirin A, phellodendrine, PHT-427, deltonin, crosstide, N-feruloyloctopamine, glaucocalyxin A, CAY 10404, polygalasaponin F, hederacolchiside A1, rotundic acid, K-80003, sophocarpine monohydrate, MPTOE028, kazonil B, batatasin Ill, 8-aminoadenosine, sennidin B, sennidin A, or any combination thereof.

33. (canceled)

34. The pharmaceutical composition of claim 30, further comprising at least one additional Pim kinase inhibitor, wherein the at least one additional Pim kinase inhibitor comprises SGI-1776, NVP-LGB321, a Pim-specific siRNA, or any combination thereof.

35. (canceled)

36. The pharmaceutical composition of claim 30, further comprising at least one mTOR inhibitor, wherein the at least one mTOR inhibitor comprises everolimus, deferolimus, gefitinib, temsirolimus, torin-1, torin-2, vistusertib, PP242, ridaforolimus, umirolimus, zotarolimus, rapamycin, a rapamycin derivative, or any combination thereof.

37. (canceled)

38. A method for treating at least one disease or disorder, the method comprising administering the compound of claim 1 to a subject.

39. The method of claim 38, wherein the disease comprises glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, oral squamous cell cancer, hepatocellular carcinoma, bladder cancer, non-small lung cancer, melanoma, or any combination thereof.

40. (canceled)

41. The method of claim 38, wherein the disorder comprises a metabolic disorder, wherein the metabolic disorder comprises hypercholesterolemia, type 1 diabetes, type 2 diabetes, gestational diabetes, metabolic syndrome, obesity, or any combination thereof.

42.-45. (canceled)
